(19) United States
(12) Patent Application Publication
(10) Pub. No.: US 2026/0341496 A1
(43) Pub. Date: Oct. 29, 2026

(54) SYSTEM, LOCATION TAG ASSEMBLY AND PATIENT SUPPORT APPARATUS FOR LOCATING MOBILE TAGS IN REAL-TIME IN A MEDICAL FACILITY

(71) Applicant: UMANO MEDICAL INC., L'Islet (CA)

(72) Inventors: Anouer KEBIR, Levis (CA); Esther BERTHELOT, Cap d'Espoir (CA); David MAYEN MORENO, L'Ancienne-Lorette (CA); Ali DUFOUR, St-Augustin-De-Desmaures (CA)

(73) Assignee: UMANO MEDICAL INC., L'Islet (CA)

(21) Appl. No.: 19/654,605

(22) Filed: Apr. 22, 2026

Related U.S. Application Data
(63) Continuation-in-part of application No. 19/462,167, filed on Jan. 28, 2026.
(60) Provisional application No. 63/796,697, filed on Apr. 29, 2025.

(51) Int. Cl. G16H 40/40; A61G 7/05; G06K 7/10; H04W 4/029; H04W 4/33; H04W 4/80

(52) U.S. Cl. CPC G16H 40/40; A61G 7/05; G06K 7/10366; H04W 4/029; H04W 4/33; A61G 2205/60; H04W 4/80

(57) ABSTRACT

There is provided a system, a patient support apparatus, and a location tag assembly for locating mobile tags in real-time in a medical facility using a plurality of wireless signal modalities where the tags could be worn by patients or attached to equipment. The location tag assembly can be secured to a patient support apparatus which could be located precisely with respect to a wireless locating node. The wireless locating nodes may be used to locate a first type of mobile tag. The location tag assembly may be used to locate a second type of mobile tag. The location information may be transmitted to a server to precisely locate patient support apparatuses, medical equipment, patients and staff in real-time.

### TECHNICAL FIELD

The present disclosure relates to a system, a location tag assembly and a patient support apparatus for locating mobile tags in real-time in a medical facility where the tags could be worn by patients or attached to equipment.

### BACKGROUND OF THE ART

Real-time localization technologies have been increasingly adopted in healthcare environments to support monitoring, tracking, and management of patients, beds, and medical equipment. These systems are generally implemented to maintain situational awareness within a clinical setting and to allow clinical and support staff to determine the physical location of assets and individuals within a defined space such as a hospital ward, intensive care unit, or surgical suite. Traditional methods for patient or equipment tracking have relied on manual data entry, visual inspection, or the use of fixed-location data collection points, which may not offer continuous or real-time positional awareness.

While such systems are available in various forms, the real-time synchronization of localization data across patients, beds, and mobile medical equipment remains subject to integration constraints and signal interference challenges in complex indoor clinical environments. Further, existing implementations may not fully address dynamic changes in room configurations, reallocation of beds, or context-aware associations between patient identity, bed occupancy, and the use of specific medical devices. Accordingly, improved systems and methods for real-time localization in hospital environments are under continued development to provide updated positional correlation across heterogeneous entities within a care facility.

There is a desire to locate equipment and patients in a medical facility.

### SUMMARY

It is an object of the present disclosure to ameliorate at least one drawback of existing systems.

It is an object of one or more implementations of the present disclosure to provide systems, methods, patient support apparatuses and non-transitory storage mediums for locating mobile tags in real-time in a medical facility, where the tags could be worn by patients and/or attached to equipment.

Modern medical facilities present a uniquely complex operational environment in which the precise, real-time localization of personnel, patients, and assets is essential to both clinical efficacy and institutional safety. Indeed, no single wireless locating technology is capable of satisfying the full spectrum of positioning requirements inherent to such an environment, given the substantial variation in required accuracy, coverage range, infrastructure constraints, power availability, and the dynamic nature of clinical workflows.

One or more implementations of the present disclosure may be implemented in a medical facility environment where patient support apparatuses may be located, for example, as part of a real-time locating system (RTLS) using fixed wireless locating nodes exchanging different types of wireless signals with patient support apparatuses and tags, such as, but not limited to, infrared (IR), ultrasonic, Bluetooth including Bluetooth Low Energy (BLE), ultrawideband (UWB) and Wi-Fi.

One or more implementations of the present disclosure enable at least approximately locating mobile tags via wireless locating nodes or patient support apparatuses without the use of pre-established signal calibration data or pre-mapped tag location information. The mobile tags may be equipped with transmitters and/or receivers of one or more types of wireless signals, such as, but not limited to, infrared (IR), Bluetooth including Bluetooth Low Energy (BLE), ultrawideband (UWB) and/or radiofrequency identification (RFID). Depending on the type of wireless technology used, the mobile tags may be located by a location engine via the wireless locating nodes, either directly (e.g., by determining wireless signal parameters such as signal strength) or indirectly (e.g., via patient support apparatuses detecting the mobile tags using proximity sensing, and the patient support apparatuses being located by the wireless locating nodes using wireless signals).

One or more implementations of the present disclosure enable reuse of an existing real-time location system (RTLS) infrastructure, where the wireless locating nodes or terminals currently installed could be operable without requiring relocation or hardware modification and by performing firmware and/or software updates. One or more implementations of the present disclosure further enable a reduction in development costs through the use of standard tags (e.g., iBeacon tags and RFID tags), thereby avoiding hardware development expenditures. By employing existing and widely available beacons, financial investments typically associated with the design and manufacturing of proprietary components are not incurred. One or more implementations of the present disclosure also provides a localization configuration with increased flexibility by using mobile tags available in multiple formats, including compact adhesive discs configured for attachment to various objects, adaptable bracelets, cards, or blocks. These available formats for mobile tags according to the present disclosure enable the system to be adapted to different localization requirements in hospital environments while maintaining flexibility of implementation.

At least some implementations of the present technology use a first modality for locating patient support apparatuses by locating the patient support apparatuses at a sub-room level or room zone level using one or more types of wireless signal exchanged with a wireless locating node. It will be appreciated that the patient support apparatuses may have wireless locating tags attached thereto or integrated to a circuitry of the apparatus. The first modality may use a combination of wireless signals to locate the beds in zones of a room. As a non-limiting example, a combination of ultrawideband signals and infrared signals may be used to determine the distance of the bed with respect to a wireless locating node with cm-grade accuracy and the presence of the bed within the same room, respectively.

At least some implementations of the present technology enable using a second modality for locating tags (which may be worn by patients and/or attached to objects) by estimating the distance using the wireless signal strength data determined by wireless nodes in the facility, and by classifying the estimated distance between nodes and tags into two or more proximity categories, such as very close, close and far. As a non-limiting example, the second modality may use Bluetooth low energy (BLE) wireless signals to determine proximity categories with 1-3 m accuracy. In one or more alternative implementations, tags may be located by estimating the distance using non-power based wireless signal parameters, such as signal propagation time measurements or phase difference measurement across multiple frequencies determined by the wireless nodes in the facility.

At least some implementations of the present disclosure enable using a third modality for locating RFID tags (which may be worn by patients, staff and/or attached to medical equipment) by using specifically designed RFID readers connected to the circuitry of the patient support apparatuses. Since the patient support apparatuses are located using a first modality, the patients, staff and/or equipment may be located with respect to the patient support apparatuses using the second and third modalities. Thus, the patient support apparatuses may be used as hubs for locating patients, medical staff and medical equipment in real-time, thereby providing an accurate real-time representation of the location of beds, patients, staff and medical equipment, which also provides security, inventory management and enable optimizing resources allocated to caregiving and improve clinical outcomes. Appropriate actions may then be executed in response to various detection criteria, such as for example triggering automated workflows including care protocols, staff notifications, safety alerts, tailored to that patient's specific needs and/or risk profile.

One or more implementations of the present disclosure provide a location tag assembly configured for using multiples electromagnetic signal modalities or domains for communication and location purposes, which may be integrated into existing RTLS and retrofit to patient support apparatuses that do not have transceivers for location purposes.

One or more implementations of the location tag assembly provides a self-contained, multi-protocol wireless module that is dimensionally and electrically configured for installation into existing patient support apparatus platforms without structural modification to the bed frame, the location tag assembly being mountable to components of commercially deployed hospital bed architectures. One or more implementations of the location tag assembly consolidates antenna feeds, power regulation, and inter-protocol arbitration circuitry into a unified assembly that interfaces with the apparatus control system by way of a standardized communication databus, thereby rendering the location tag assembly operable with legacy bed platforms whose original configurations did not include wireless localization capability, which also eliminates the need for separate installation, wiring, and commissioning of discrete single-protocol modules, reducing installation time and the number of discrete components subject to independent failure.

One or more implementations of the RTLS system of the present disclosure enable cooperation and fusion of multiple electromagnetic location signals by resolving the positional ambiguity inherent to any single wireless protocol estimate by applying complementary measurement geometries, which enables to augment the capabilities provided by a single protocol (e.g., RFID or Bluetooth alone). Further, some implementations of the present disclosure also provide a multi-protocol architecture that is redundant and, in some cases, provides operational continuity in the event of degradation or absence of any individual wireless modality.

It will be appreciated that the patient support apparatus equipped with the location tag assembly in a RTLS system acts as a hub by combining its own positional and operational state (e.g., occupied/unoccupied, bed-exit alert active, siderail configuration) with the localization state of associated tags, is operable to generate context-dependent outputs conditioned on spatial relationships between the patient support apparatus and detected entities (i.e., patients, staff and equipment), including nurse proximity alerts, equipment-to-bed association events, and patient-wander detection triggers, without reliance on external computing infrastructure.

It will be appreciated that the location information may be communicated with servers, nurse call systems, staff computing devices, electronic medical records computers, and admission, discharge and transfer (ADT) systems and the like.

In accordance with a broad aspect of the present disclosure, there is provided a real-time locating system (RTLS) for use in a medical facility, the RTLS comprising: a location tag assembly adapted to be mounted to a patient support apparatus, the location tag assembly comprising a first set of wireless transceivers comprising a first wireless transceiver, a second wireless transceiver, at least one radiofrequency identification (RFID) transceiver, and a third wireless transceiver, the location tag assembly being configured for: determining, using the first wireless receiver and the second wireless transceiver, a distance and a location of the location tag assembly in an area, transmitting, using the third wireless transceiver, the location and the distance to a computing device, detecting, using the at least one RFID transceiver, a RFID tag by receiving a response RFID signal therefrom, determining, based on the response, an identifier (ID) of the RFID tag, and transmitting, using the third wireless transceiver, the identifier of the RFID tag, a wireless locating node positioned in the area, the wireless locating node comprising a second set of wireless transceivers comprising a corresponding first wireless transmitter and a corresponding second wireless transceiver, the wireless locating node being configured for: exchanging signals with the location tag assembly using the corresponding first wireless transmitter and the corresponding second wireless transceiver to enable the location tag assembly to determine the distance and location, a computing device connected to the location tag assembly, the computing device being configured for receiving, from the location tag assembly using the third wireless transceiver, the distance and the location of the location tag assembly relative to the wireless locating node, associating the location tag assembly with a location of the wireless locating node in the medical facility to thereby obtain a real-time clinical location of the location tag assembly, receiving, from the location tag assembly using the third wireless transceiver, an identifier of the RFID tag, determining, based on the identifier of the RFID tag, an entity associated with the RFID tag, associating the entity associated with the RFID tag to the location of the location tag assembly, and outputting, to a user interface, an indication of a location of the location tag assembly and the entity associated with the RFID tag.

In one or more implementations of the RTLS system, the wireless locating node further comprises a Bluetooth transceiver, the wireless locating node being configured for determining signal parameters of a Bluetooth signal broadcast by a Bluetooth tag, and the computing device is further configured for: determining, using the signal parameters of the Bluetooth signal, a proximity of the Bluetooth tag to the wireless locating node, and determining another entity associated with the Bluetooth tag, and outputting to a user interface, an indication of the proximity of the other entity to the wireless locating node.

In one or more implementations of the RTLS system, the at least one RFID transceiver is adapted to be mounted below a deck panel of the patient support apparatus.

In one or more implementations of the RTLS system, the location tag assembly is removably secured to a barrier of the patient support apparatus.

In one or more implementations of the RTLS system, the at least one RFID transceiver is positioned to face a patient support surface of the patient support apparatus when the location tag assembly is secured to the barrier of the patient support apparatus.

In one or more implementations of the RTLS system, the first wireless transceiver comprises an infrared receiver and the corresponding first wireless transmitter comprises an infrared transmitter, the second wireless transceiver and the corresponding second wireless transceiver each comprise a respective ultrawideband transceiver, and the third wireless transceiver comprises a Wi-Fi transceiver.

In one or more implementations of the RTLS system, the entity comprises one of: a patient, a staff member, and a medical equipment.

In one or more implementations of the RTLS system, the computing device is further configured for: transmitting a signal to cause activation of an alarm in response to the entity being associated with the location.

In accordance with a broad aspect of the present disclosure, there is provided a patient support apparatus comprising: a frame, a patient support surface mounted to the frame, and a controller mounted to at least one of the frame and the patient support surface, a location tag assembly operatively connected to the controller, the location tag assembly comprising: a first wireless receiver configured for receiving a first type of wireless signal, a second wireless transceiver configured for transmitting and receiving a second type of wireless signal, at least one radiofrequency identification (RFID) transceiver configured for transmitting and receiving RFID signals for detecting RFID tags, a third wireless transceiver configured for transmitting a third type of wireless signal, the controller being configured for: determining, using the first wireless receiver and the second wireless transceiver, a distance and presence of the patient support apparatus relative to a wireless locating node, transmitting, using the third wireless transceiver, the location and the distance to a computing device, detecting, using the at least one RFID transceiver, a RFID tag by receiving a response RFID signal therefrom, determining, based on the response, an identifier (ID) of the RFID tag, and transmitting, using the third wireless transceiver, the identifier of the RFID tag to a computing device.

In one or more implementations of the patient support apparatus, the RFID transceiver comprises at least one of: a circular polarization RFID antenna and ultrahigh frequency (UHF) RFID antenna.

In one or more implementations of the patient support apparatus, the patient support apparatus comprises a removable footboard selectively couplable to a foot end of the frame, the location tag assembly being located within the removable footboard.

In one or more implementations of the patient support apparatus, the first wireless receiver comprises an infrared (IR) receiver, the second wireless transceiver comprises an ultrawideband (UWB) transceiver, and the third wireless transceiver comprises at least one of: a Wi-Fi transceiver and a Bluetooth transceiver.

In one or more implementations of the patient support apparatus, the location tag assembly comprises a top board disposed above a bottom board and operatively connected thereto, the top board facing a patient support surface of the patient support apparatus when the removable footboard is coupled to the patient support apparatus, the top board comprising an RFID antenna.

In one or more implementations of the patient support apparatus, the bottom board comprises the UWB transceiver, the IR receiver and the Wi-Fi transceiver, the IR transceiver facing away from the patient support surface when the removable footboard is coupled to the patient support apparatus.

In one or more implementations of the patient support apparatus, the removable footboard comprises at least one connector for operatively connecting the location tag assembly to a databus and a power circuit of the patient support apparatus.

In accordance with a broad aspect of the present disclosure, there is provided a location tag assembly adapted to be mounted to a patient support apparatus, the location tag assembly comprising: a first wireless receiver configured for receiving a first type of wireless, a second wireless transceiver configured for transmitting and receiving a second type of wireless signal, at least one radiofrequency identification (RFID) transceiver configured for transmitting and receiving RFID signals for detecting RFID tags, a third wireless transceiver configured for transmitting a third type of wireless signal, at least one processing unit operatively connected to the first wireless receiver, the second wireless transceiver, the third wireless transceiver and the at least one RFID transceiver, the at least one processing unit being configured for: determining, using the first wireless receiver and the second wireless transceiver, a distance and presence of the patient support apparatus relative to a wireless locating node, transmitting, using the third wireless transceiver, the location and the distance to a computing device, detecting, using the at least one RFID transceiver, a RFID tag by receiving a response RFID signal therefrom, determining, based on the response, an identifier (ID) of the RFID tag, and transmitting, using the third wireless transceiver, the identifier of the RFID tag to a computing device.

In one or more implementations of the location tag assembly, the first wireless receiver comprises an infrared (IR) receiver, the second wireless transceiver comprises an ultrawideband (UWB) transceiver, and the third wireless transceiver comprises at least one of: a Wi-Fi transceiver and a Bluetooth transceiver.

In one or more implementations of the location tag assembly, the location tag assembly comprises a top board disposed above a bottom board and operatively connected thereto, the top board facing a patient support surface when the location tag assembly is coupled to the patient support apparatus, the top board comprising an RFID antenna.

In one or more implementations of the location tag assembly, the bottom board comprises the UWB transceiver, the IR receiver and the Wi-Fi transceiver, the IR receiver facing away from the patient support surface when the removable footboard is coupled to the patient support apparatus.

In one or more implementations of the location tag assembly, the location tag assembly comprises at least one connector for operatively connecting the location tag assembly to a databus and a power circuit of the patient support apparatus.

In one or more implementations of the location tag assembly, the RFID transceiver comprises at least one of: a circular polarization RFID antenna and ultrahigh frequency (UHF) RFID antenna.

In one or more implementations of the location tag assembly, the IR receiver comprises a 140-degree wide angle receiver.

Each of the methods described herein may be implemented as computer-readable instructions stored in a non-transitory storage medium, which may be executed by one or more processing units.

In the context of the present specification, a “server” is a computer program that is running on appropriate hardware and is capable of receiving requests (e.g., from electronic devices) over a network (e.g., a communication network), and carrying out those requests, or causing those requests to be carried out. The hardware may be one physical computer or one physical computer system, but neither is required to be the case with respect to the present technology. In the present context, the use of the expression a “server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “electronic device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that an electronic device in the present context is not precluded from acting as a server to other electronic devices. The use of the expression “an electronic device” does not preclude multiple electronic devices being used in receiving/sending, carrying out or causing to be carried out any task or request, or the consequences of any task or request, or steps of any method described herein. In the context of the present specification, a “client device” refers to any of a range of end-user client electronic devices, associated with a user, such as personal computers, tablets, smartphones, and the like.

In the context of the present specification, the expression “computer-readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e. its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication. As a non-limiting example, to “display an indication” involves rendering, through execution of instructions, at least one visual element representing the information element, which may be the information element itself (e.g., value or text) or a graphical element representing the information element (e.g. progress bar or color for intensity values).

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first server” and “third server” is not intended to imply any particular order, type, chronology, hierarchy or ranking (for example) of/between the server, nor is their use (by itself) intended to imply that any “second server” must necessarily exist in any given situation. Further, as discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements described herein and/or shown in the figures, including a “processor”, “processing unit”, “graphics processing unit” or the like may be provided through the use of dedicated hardware as well as hardware capable of executing software in association with appropriate software. When provided by a processing unit, the functions may be provided by a single dedicated processor, by a single shared processor, or by a plurality of individual processors, some of which may be shared. In some non-limiting implementations of the present technology, the processing unit may be a general purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, a microcontroller, network processor, an integrated circuit (IC) purposed for specific operations within an embedded system, application specific integrated circuit (ASIC), field programmable gate array (FPGA), a system on a chip (SoC), and the like.

The functions of the various elements described herein and/or shown in the figures including “memories” may include volatile and non-volatile computer-readable storage media realized in semiconductor, magnetic, optical, or other physical forms and may reside locally, be distributed across multiple devices, or be accessed through a network-based storage service. In particular implementations, a given one of the memories comprises volatile storage such as random-access memory (RAM), static RAM (SRAM), or dynamic RAM (DRAM), and/or non-volatile storage such as flash memory, electrically erasable programmable read-only memory (EEPROM), ferroelectric RAM (FRAM), magnetic disk, optical disk, or solid-state drive. The memories may be removable or non-removable, may interface with the processors through one or more buses or interconnects employing standard or proprietary protocols, and are configured to store computer-readable instructions, data structures, configuration parameters, machine-learning models, and other information processed by the processors to carry out the functionalities described herein.

Software modules, or simply modules which are implied to be software, may be represented herein as any combination of flowchart elements or other elements indicating performance of process steps and/or textual description. Such modules may be executed by hardware that is expressly or implicitly shown.

### BRIEF DESCRIPTION OF THE DRAWINGS

Having thus generally described the nature of the technology, reference will now be made to the accompanying drawings, showing by way of illustration example implementations thereof and in which:

FIG. 1 is a perspective view of a patient support apparatus in accordance with one or more non-limiting implementations of the present disclosure;

FIG. 2 is a schematic diagram of a real-time localization communication system 200 in accordance with one or more non-limiting implementations of the present disclosure;

FIG. 3 is a schematic diagram of an environment implementing an RTLS system in a medical facility in accordance with one or more non-limiting implementations of the present disclosure;

FIG. 4 is a schematic diagram of an environment in a medical facility where different locations of mobile tags attached to patients or equipment are shown in accordance with one or more non-limiting implementations of the present disclosure;

FIG. 5 illustrates a schematic diagram of an electrical circuitry of the hospital bed connected to a location tag assembly in accordance with one or more non-limiting implementations of the present disclosure;

FIG. 6 is a flow chart of steps of a first procedure for locating mobile tags in accordance with one or more non-limiting implementations of the present disclosure;

FIG. 7 is a flow chart of main steps of a procedure for connecting to a message queuing telemetry transport (MQQT) broker in accordance with one or more non-limiting implementations of the present disclosure;

FIG. 8 is a schematic diagram of a location system using RFID in accordance with one or more non-limiting implementations of the present disclosure;

FIG. 9A is a perspective view of a location tag assembly fixed to a housing that is insertable inside a removable footboard of a hospital bed in accordance with a non-limiting implementation of the present disclosure;

FIG. 9B is a front view of the location tag assembly on the housing showing the RFID antenna;

FIG. 9C is a bottom view of the location tag assembly and housing of FIG. 9B.

FIG. 10A is a front view of an outer surface of the top board of the location tag assembly, the top board including a RFID antenna in accordance with a non-limiting implementation of the present disclosure;

FIG. 10B is a front view of an inner surface of the bottom board of the location tag assembly of FIG. 9A;

FIG. 10C is a front view of an outer surface of the top board of the location tag assembly of FIG. 9A;

FIG. 11 shows a perspective view of a bed with a representation of an approximate left RFID zone and an approximate right RFID zone with respective left and right RFID readers under a seat panel in accordance with a non-limiting implementation of the present disclosure;

FIG. 12 shows a perspective view of a bed with a representation of an approximate main detection RFID zone and a secondary RFID detection zone and an approximate right RFID zone 1210 with respective left and right RFID readers (not numbered) under the seat panel.

FIG. 13A is a perspective view of a central panel of a hospital bed taken from below showing a left side RFID reader and right side RFID reader operatively connected to a controller;

FIG. 13B is a perspective view of a patient wearing a RFID wristband in a hospital bed;

FIG. 14A is a schematic diagram of a first configuration of a hospital bed with an integrated location tag assembly operatively connected to a board and a RFID reader in accordance with a non-limiting implementation of the present disclosure;

FIG. 14B is a schematic diagram of a second configuration of a hospital bed with a retrofitted location tag assembly in accordance with a non-limiting implementation of the present disclosure;

FIG. 15 is a flowchart of a method of locating wireless mobile tags using power-based signal parameters in accordance with one or more non-limiting implementations of the present disclosure;

FIG. 16 is a flowchart of a method of locating wireless mobile tags using one of signal propagation time measurement parameters or phase difference measurement across frequencies parameters in accordance with one or more non-limiting implementations of the present disclosure;

FIG. 17 is a flowchart of a method of locating mobile tags comprising a first set of BLE mobile tags and second set of RFID mobile tags in accordance with one or more non-limiting implementations of the present disclosure;

### DETAILED DESCRIPTION

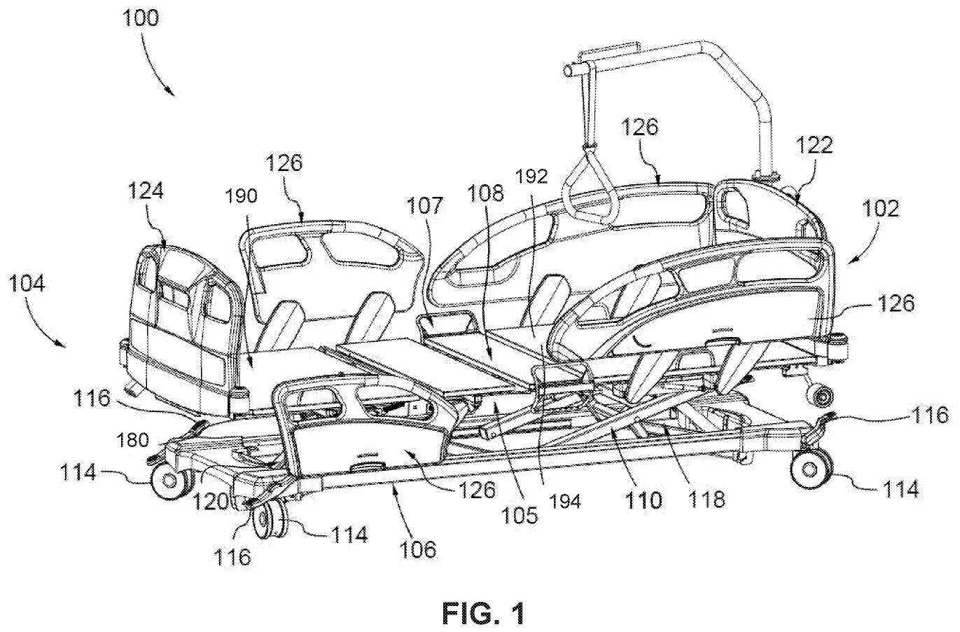

A patient support apparatus 100 in accordance with an embodiment of the present technology is illustrated in FIG. 1. The patient support apparatus 100 may be used in a medical setting for supporting a patient. In this embodiment, the patient support apparatus 100 is a hospital bed 100 that is used in a hospital, and in particular in an intensive care unit (ICU) or medical-surgical (med-surg) setting. It is contemplated that, in other implementations, the hospital bed 100 may be a different type of patient support apparatus such as, for example, a stretcher, a motorized chair, an operating room table, or other specialty tables (e.g., an examination table) used in different contexts.

With reference to FIG. 1, the bed 100 has a head end 102 and a foot end 104 opposite each other and defining a length of the bed 100 therebetween. As will be appreciated, in use, when the patient is lying on the bed 100, the patient's head is closer to the head end 102 while the patient's feet are closer to the foot end 104. The bed 100 also has left and right sides 105, 107 extending between the head end 102 and the foot end 104 and defining a width of the bed 100 therebetween.

Some of the structural components of the bed 100 will be designated hereinafter as “right”, “left”, “head” and “foot” from the reference point of an individual lying on their back on the bed 100 with their head oriented toward the head end 102 of the bed 100 and their feet oriented toward the foot end 104 of the bed 100. Similarly, the term “headward” refers to an element located towards the head end 102 of the bed 100 and the term “footward” refers to an element located towards the foot end 104 of the bed 100. Furthermore “interior” and “exterior” views are also designated from the reference point of the patient lying in the bed. Therefore, an interior view shows an element as seen by the patient looking toward the environment outside of the bed and an exterior view shows an element as seen by a person outside of the bed. Generally, an exterior view shows the exterior surfaces of the bed, and the interior view shows the interior surfaces of the bed. The terms “inner” and “outer” will similarly be used to describe the position of elements relative to the bed.

The bed 100 has a base 106 and a patient support assembly 108 operatively connected to the base 106. Four casters 114 are connected to the base 106 at respective corners thereof to allow the bed 100 to be moved along a floor. Additional casters may be provided in other implementations. Respective brakes 116 are provided for each caster 114 to selectively lock and unlock the casters 114. The bed 100 may also have a drive wheel (not shown) connected to the base 106 for driving the bed 100 on the floor.

The patient support assembly 108, which is disposed above the base 106, is configured to accommodate the patient thereon and includes an upper frame 180 and a deck 190 supported by the upper frame 180. The upper frame 180 extends from a head end 201 to an opposite foot end 203. The upper frame 180 has left and right longitudinal rails 202 that extend parallel to each other and are interconnected by a plurality of transversal members 204a, 204b, 204c extending therebetween. The upper frame 180 may be configured in any suitable manner in other implementations.

The deck 190 defines a top face on which a support surface such as a mattress or the like is supported. The deck 190 includes a plurality of deck sections that are connected to the frame 180 and, in use, support different areas of the patient's body. Notably, the deck sections forming the deck 190 include an upper body deck section (not numbered) for supporting an upper body of the patient, a seat deck section (not numbered) for supporting the patient's upper legs area, a thigh deck section (not numbered) for supporting the patient's thighs, and a foot deck section (not numbered) for supporting the patient's lower legs and feet. The deck sections (not numbered) are longitudinally consecutive to each other and together form the top face. Notably, the upper body deck section, seat deck section, thigh deck section and foot deck section have an upper body deck section panel, a seat deck section panel, a thigh deck section panel and a foot deck section panel, respectively. The deck section panels define respective top faces such that, together, they form the top face 192 of the deck 190.

The deck sections can be articulated in such a manner as to optimize comfort for the patient lying on the bed 100. The deck sections are movably connected to each other. Example implementations for the articulation of deck sections are known in the art and some are described in greater detail in International Patent Application Publication No. WO2024154074, by the same Applicant, the entirety of which is incorporated by reference herein. It is contemplated that fewer deck sections may be provided in other implementations (e.g., three deck sections).

The bed 100 has a plurality of barriers generally disposed around the patient support assembly 108 including endboards, namely a headboard 122 at the head end 102 and a footboard 124 at the foot end 104, and at least one siderail 126 on the left and right sides 105, 107. The siderails 126 can be raised to prevent the patient from falling off the bed 100 or lowered (as shown for one of the siderails 126 in FIG. 1) to allow the patient to exit the bed 100 and/or to allow care providers to access and attend to the patient. In the embodiment shown in FIG. 1, the siderails 126 include two siderails on each side (a head siderail and a foot siderail).

In some implementations, the footboard 124 is removably securable to the bed 100. The footboard 124 comprises a body panel and defines an outward facing surface and an inward facing surface. In some implementations, the body panel of the footboard 124 may include a user interface (not numbered) on the outward facing surface to enable a caregiver to control functions of the bed 100. The footboard 124 may include handles (not numbered) to facilitate moving the hospital bed 100 by the caretaker from one place to another. The footboard 124 further includes engagement interfaces for coupling the footboard 124 to the bed 100. The engagement interfaces include mechanical components to removably secure the footboard 124 to the bed and electrical components to electrically connect the footboard 124 to the power circuit and components of the bed 100.

In some implementations, the footboard 124 may include a plate that enables accessing the interior cavity of the body panel. In some implementations, the plate enables securing additional components to the bed 100, such as sensors, and communication and computing components, such as a location tag assembly (not shown in FIG. 1), as will be explained hereinafter.

An elevation system 110 operatively connects the patient support assembly 108 to the base 106 to move the patient support assembly 108 relative to the base 106, namely selectively raising or lowering the patient support assembly 108 relative to the base 106. In addition to raising and lowering the patient support assembly 108, the elevation system 110 also allows the bed 100 to assume various different positions such as a flat horizontal position (shown in FIG. 1), a Trendelenburg position, a reverse Trendelenburg position, a cardiac chair position and a full chair position. It is contemplated that, in other implementations, the bed 100 may be configured to assume more or fewer positions. For instance, in some cases, the bed 100 may permanently remain in the flat horizontal position and simply be raised and lowered by the elevation system 110.

The base 106 further comprises force sensors for determining forces exerted on the bed 100. In one example embodiment, a plurality of load cells are adapted to provide an indication of the weight on the bed 100. The one or more load cells are operatively connected to a controller. The force sensors may also be used for determining a position of the patient in the bed 100, a center of mass of the weight present on the bed, a level of activity of the patient and a load distribution of the weight of the patient.

In one or more implementations, the bed 100 includes four load cells, where each load cell is positioned at a respective corner i.e., foot right (FR), foot left (FL), head right (HR) and head left (HL). Each load cell is operable to generate a signal comprising a voltage value upon presence of a weight on the bed, which is converted into a respective weight value for each load cell received by the controller. The respective weight values may be used to determine the weight of the person or the object(s) on the bed 100, a position and/or movement of the center of gravity on the bed 100, and/or further processed to determine patient agitation and activity, vital signs (e.g., heart rate (HR) and respiratory rate (BR)) and the like. In one or more alternative implementations, the bed 100 may include a different number of load cells, such as a single load cell.

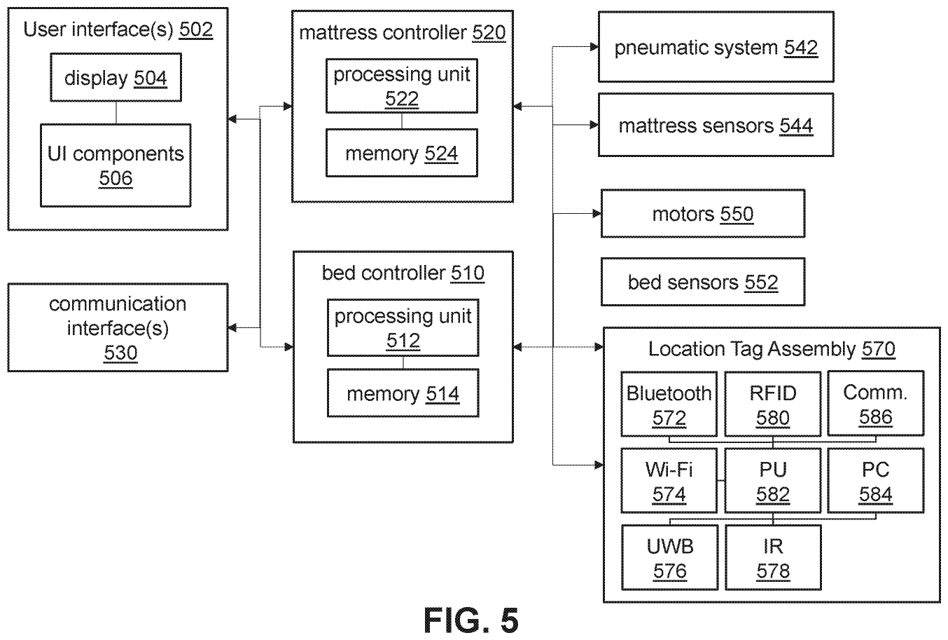

With reference to FIG. 5, there is shown a schematic diagram of an electrical circuitry 500 of a patient support apparatus in accordance with non-limiting implementations of the present technology.

The electrical circuitry 500 is a simplified schematic diagram of components and subsystems of the patient support apparatus (implemented as the hospital bed 100 of FIG. 1) and a patient support surface (implemented as a mattress).

It should be appreciated that alternative arrangements of the components and subsystems may be used without departing from the scope of the present technology.

The electrical circuitry 500 includes a user interface 502, a bed controller 510, a tag assembly 560, communication interface(s) 530 and various motors 550 and bed sensors 552 for monitoring and controlling various functions of the hospital bed 100. The mattress (not shown in FIG. 1) is operatively connected to the hospital bed 100 and includes a pneumatic system 542 and sensors 544 operatively connected to a mattress controller 520. The mattress controller 520 is operatively connected to the bed controller 510.

The user interface 502, the bed controller 510, the mattress controller 520 and the tag assembly 560 are electrically connected to each other and communicate via one or more databuses. In one or more implementations, the user interface 502, the bed controller 510, the mattress controller 520 and the tag assembly 560 may communicate via a local interconnected network (LIN) databus protocol.

### Control Panel

The user interface 502, also referred to as control interface, control panel, and input device, enables a user (e.g., caregiver) to interact with the different components and subsystems of the hospital bed 100 and/or mattress by displaying information, receiving user inputs and transmitting instructions to control different components and functions of the hospital bed 100 and/or mattress.

The user interface 502 may be mounted on or may be adjacent to the hospital bed 100. It will be appreciated that one or more control panels with different components may be mounted on barriers such as siderails 126, end board (headboard 122, footboard 124) and/or fixed to components of the hospital bed 100. It will be appreciated that the user interface 502 may be provided at different locations, may be integrated into the bed or may be a separate device operatively connected to at least one component of the bed 100.

The user interface 502 includes various user-interface (UI) components such as, but not limited to, one or more of: visual components (e.g., displays and lights), audio components (e.g., speakers and microphones), control or input components (e.g., buttons, knobs, switches, keypads, feedback components and touchscreens), sensors (e.g., proximity sensors) and additional input/output interfaces and communication components (e.g., connectivity and charging ports, wireless connectivity modules). As shown schematically in FIG. 5, the user interface 502 includes a display 504 and UI components 506.

The one or more displays (shown as display 504 in FIG. 5), also referred to as display screen(s), display device(s), display unit(s) or display interface(s), may include one or more of a liquid crystal display (LCD), light emitting diode (LED) display, organic light emitting diode (OLED) display, plasma displays, e-ink (electronic Ink) display, touch screen displays, quantum dot displays, digital light processing (DLP) projector, head-up displays (HUD), virtual reality (VR) headset displays, and augmented reality (AR) displays. It will be appreciated that in alternative implementations of the present technology, the display 504 may be optional. As a non-limiting example, a display 504 may be mounted on the exterior faces of foot end siderails 156.

In one or more implementations, the user interface 502 is configured to display graphical user interfaces (GUIs) to control and/or view different data relating to the hospital bed 100, the mattress and/or the patient.

A given user may interact with the GUIs displayed on the user interface 502 by using the display 504 (e.g., if implemented as a touchscreen), the UI components 506 (e.g. button, knobs, switches), voice, and/or gestures.

The user interface 502 is configured for receiving data signals from, and transmitting data signals to the bed controller 510 and/or the mattress controller 520.

The received data may include for example feedback and error notifications. The feedback is transmitted from the controllers 510, 520 over the communication pathway and received by the user interface 502 to update its display or alert the user.

In some implementations, user interfaces may be provided on the interior faces of the siderails 126 and/or endboards 122, 124 of the bed 100 to enable the patient to interact with the bed (“patient control panel”), while control panels provided on the exterior faces of the siderails and/or endboards of the bed 100 may be provided to enable personnel to interact with the bed 100. As a non-limiting example, the patient user interfaces may have different components, UI elements, and/or accessible functionalities to prevent patients from controlling features of the bed reserved for the appropriate care personnel (e.g., disabling bed exit or other functionalities) that may be controllable using the panels located on the exterior faces (e.g., siderail and footboard).

### Controllers

The hospital bed 100 includes the bed controller 510 and the mattress includes the mattress controller 520.

In some implementations, the bed controller 510 and the mattress controller 520 may be implemented as a single controller. In one or more other implementations, the user interface 502 may be connected to additional controllers or and the control functionalities of the bed and mattress may be distributed among a plurality of controllers.

The bed controller 510 is operatively connected to various motors 550 and bed sensors 552 and components of the hospital bed 100, which may include, as a non-limiting example, adjustment mechanisms, actuators, sensors, integrated weighing systems, alert indicators, and the like. In one or more implementations, the bed controller 510 may be integrated to or attached to the hospital bed 100.

In one or more implementations, the bed controller 510 is configured to read data published on the databus (e.g. local interconnect network (LIN) bus) by the components of the electrical circuitry 500 and publish data on the databus.

The bed controller 510 includes at least a processing unit 512 and a memory 514. Additionally, the bed controller 510 may include or be operatively connected to one or more input/output interfaces and communication interfaces.

The mattress controller 520 is operatively connected to components of the pneumatic system 542 and mattress sensors 544. The pneumatic system 542, may include, as a non-limiting example, blowers, pressure sensors, valves, blowers and the like being part of, for example, the pneumatic control assembly (not shown). The mattress controller 520 is configured to receive inputs (e.g., from the user interface 502 or the bed 100), process the inputs and provide outputs (e.g., to the user interface 502, to the hospital bed 100, to the pneumatic system 542 components or to other computing devices). In some implementations, the outputs may be provided as output signals. In one or more implementations, the mattress controller 520 may be located within the mattress. In other implementations, the mattress controller 520 may be located outside of the mattress, such as within or mounted to the hospital bed 100.

The mattress controller 520 includes at least a processing unit 522 and a memory 524. Additionally, the mattress controller 520 may include or be operatively connected to one or more input/output interfaces and communication interfaces (not shown).

It should be understood that the bed controller 510 and mattress controller 520 may have different types and number of components.

In one or more implementations, the processing units 512, 522, 582 which may also be referred to as processing devices, or processors, may each include a single-core microprocessor. In one or more other implementations, a given one of the processing units 512, 522, 582 may include a multi-core microprocessor. In one or more alternative implementations, a given one of the processing units 512, 522, 582 may include one or more of: a microcontroller, a digital signal processor (DSP), an integrated circuit purposed for specific operations within an embedded system, a microprocessor, a system on a chip (SoC), a field-programmable gate array (FPGA), and an application-specific integrated circuit (ASIC) configured to carry out the processing and functionalities described herein.

The memories 514, 524 may include volatile and non-volatile memories. In one or more implementations, a given one of the memories 514, 524 may include volatile memory, such as random-access memory (RAM), and/or alternatively static random-access memory (SRAM) or dynamic random-access memory (DRAM). In one or more implementations, a given one of the memories 514, 524 may include non-volatile memory, such as flash memory and/or alternatively electrically erasable programmable read-only memory (EEPROM) or ferroelectric RAM (FRAM). The memories 514, 524 are configured to store computer-readable instructions executable by the processing units 512, 522 to carry out the processing and functionalities described herein.

In one or more implementations, the bed controller 510 and the mattress controller 520 may implement respective monitoring functionalities to monitor states and components of the bed 100 via bed sensors 552 and to monitor states and components of the mattress via mattress sensors 544.

Example states and components include an indication of the elevation of the bed from the floor (hi-low adjustment) as a distance from the floor or as a minimum or maximum, the length of the bed and/or the mattress, the width of the bed and/or the mattress, the angles of the head, back, seat, thigh, leg and foot decks, rests or panels, the position (raised, lowered) of each siderail or of combinations of siderails (foot/head or left side/right side or all four siderails), the presence of endboards (headboard, footboard) in their sockets, the command and/or the angle of the tilt of the deck for foot or head elevation (Trendelenburg and Reverse Trendelenburg Adjustment), the command and/or the angle of the tilt of the deck for left or right side elevation (lateral tilting), the head-of-bed (HOB) angle (absolute angle from the horizontal which can be a combination of a backrest angle and a Trendelenburg or Reverse Trendelenburg position), the activation of a lock for the movement of the deck or the elevation, a change in position of the combined bed and mattress (flat horizontal position, Trendelenburg position, a reverse Trendelenburg position, a cardiac chair position and full chair position), the detected weight on the bed, the distribution of the weight on the bed, a change in detected weight on the bed, the presence of the patient in a lateral or longitudinal zone of the bed, a change in the level of detection of the bed exit, a maximum duration for the in bed time, a level of activity of the patient in the bed, a change in the volume of audible alerts, a change in the configuration of messages displayed on the user interface, the inflation status of each zone of the mattress, the level of immersion of the patient on the mattress, the activation, deactivation, duration or settings of specific functions of the mattress, such as percussion/vibration, alternating pressure, turn assist, continuous lateral rotation therapy (CLRT), prone therapy, side exit assistance, max inflate, full deflate, vacuum and low air loss, the level of humidity under the patient, the presence of an IV drip pole in its socket, the presence of an oxygen cylinder in its bracket, the presence of a patient lift, the presence of a medical equipment on the bed, the connection of a personal computing device to the patient power source of the bed, the connection of an external computing device to the communications network of the bed, etc.

The one or more communication interface(s) 530 may include wired and wireless communication interfaces to connect the components of the hospital bed 100 to other medical devices, computing devices (e.g., wireless modules, nurse station computer, server(s) and mobile devices), and communication networks (e.g., hospital network or cellular network) to transmit an/or receive data. The communication interface(s) 530 may include wired and/or wired communication interfaces such as, but not limited to, USB (Type-A, Type-B, Type-C), RS-232, RS-585, CAN bus, Ethernet (RJ45), Wi-Fi, Bluetooth (Classic and BLE), Zigbee, Z-Wave, RFID, NFC, infrared transceivers, audio jacks, nurse call connectors, proprietary connectors, HDMI, DisplayPort, and PCIe or edge connectors. The communication interface(s) 530 are configured for wired or wireless data exchange with external devices, control systems, monitoring equipment, or hospital networks.

The bed controller 510 and/or the mattress controller 520 are operatively connected to the location tag assembly 570.

In the context of the present disclosure, the location tag assembly 570 includes a plurality of components operatively connected to each other and distributed on one or more circuit boards (e.g. printed circuit boards (PCBs)). The location tag assembly 570 includes one or more processing units 582, a plurality of communication interfaces 586 and a power management circuit 584. The location tag assembly 570 may be connected to the bed controller 510 of the patient support apparatus via a Local Interconnect Network (LIN) bus. Additionally, or alternatively, the location tag assembly 570 may be connected via another type of databus, such as a controller area network (CAN) bus. In some implementations, the location tag assembly 570 may be powered by a battery. In other implementations, the location tag assembly 570 is powered by the power circuit of the patient support apparatus.

In one or more implementations, the location tag assembly 570 comprises a Wi-Fi module 574, a Bluetooth module 572, an ultrawideband (UWB) module 576, an infrared (IR) receiver 578, a RFID module 580 and optionally an ultrasound transceiver (not shown). The location tag assembly 570 may be used as part of a RLTS to locate the patient support apparatus, and to locate tags and beacons that may be attached to users and/or medical equipment using wireless technologies. It will be appreciated that the wireless transceivers antennas on the location tag assembly 570 or connected to the location tag assembly 570 are generally positioned to minimize interference and to maximize wireless signal detection. As used in the present context, the expression “module” refers to any functional subsystem or assembly, whether constituted by a single integrated circuit, multiple discrete components, a chipset, a circuit board assembly, or any combination thereof, that is designed to perform one or more specified functions, including all supporting circuitry, memory, processors, signal conditioning elements antennas, antenna arrays, and antenna feed structures integrated therewith or in communication therewith.

It will be appreciated that at least some of the Wi-Fi module 574, the Bluetooth module 572, the ultrawideband (UWB) module 576, the infrared (IR) receiver 578, the RFID module 580 may be combined and/or provided into more components, including separate antennas. It should be understood that at least some of the Wi-Fi module 574, the Bluetooth module 572, the ultrawideband (UWB) module 576, the infrared (IR) receiver 578, the RFID module 580 may have dedicated processing units, circuits, antennas or may be orchestrated by processing unit 582.

In one or more implementations, the location tag assembly 570 may be provided in a housing that can be removably secured to the bed 100, for example mounted to a headboard, footboard, siderail or mattress, or secured below a panel of the patient support surface using fastening elements such as bolts, screws, snap-fit connectors, adhesives and the like.

In some implementations, the location tag assembly 570 may be provided within a removable component of the hospital bed 100. The removable component may be a removable barrier, such as one of: headboard, footboard, and side rails.

In some implementations, the location tag assembly 570 is provided in a housing or mounting structure that can be inserted into the removable barrier of the patient support apparatus, for example the footboard. While reference will be made to a footboard in the following paragraphs, it should be understood that the location tag assembly 570 may be mounted into another barrier of the patient support apparatus, such as a headboard and siderails.

In some implementations, the location tag assembly 570 may be integrated into the main circuitry of the hospital bed 100. In one or more other implementations, the location tag assembly 570 may be a separate device that may be operatively connectable to the circuitry of the hospital bed 100 and that may be fixed to the hospital bed 100 (e.g., below panels of the hospital bed).

The bed 100 has many other features that will not be described in detail herein as they are well known in the art. The above description of the bed is thus not meant to be exhaustive but rather to provide sufficient context for the reader.

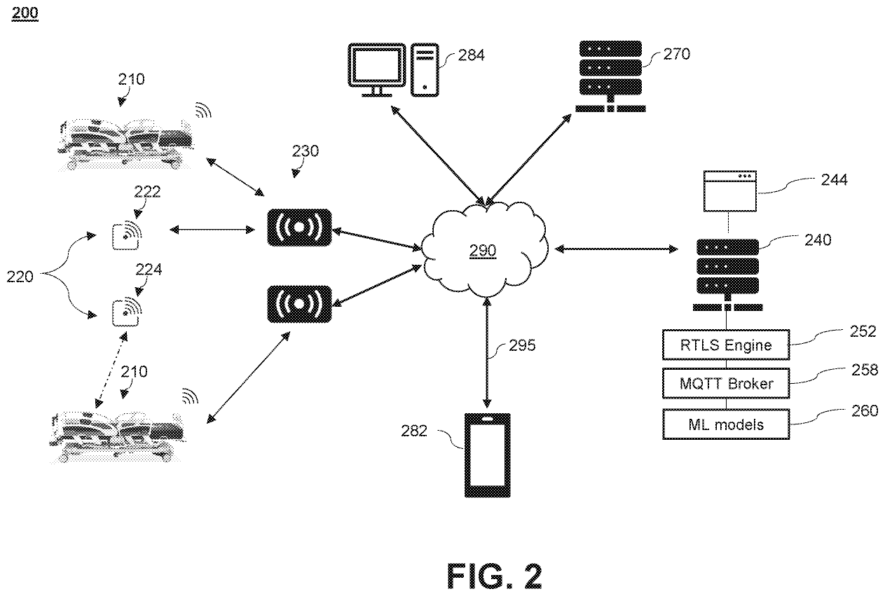

FIG. 2 illustrates a schematic diagram of a real-time localization communication system 200 suitable for implementing one or more implementations of the present technology.

The real-time localization communication system 200 comprises a plurality of patient support apparatuses 210, a plurality of mobile wireless tags 220, a plurality of wireless locating nodes 230, a workstation computer 284, one or more client devices 282, one or more servers 240 and one or more other servers 270 connected over a communication network 290 via respective communication links 295.

### Patient Support Apparatuses

The plurality of patient support apparatuses 210 (not separately numbered) may be located in a healthcare/medical facility. A given patient support apparatus 210 may be for example located within a zone of a room, a hallway, an intensive care unit, an emergency room, an operating room, and the like. The patient support apparatus 210 could be used in various locations without departing from the scope of the present technology.

A given patient support apparatus 210 includes a location tag assembly (e.g., location tag assembly 570 of FIG. 5) with communication interfaces with one or more transmitters and/or receivers configured to transmit and/or receive various types of wireless signals. In one or more implementations, the different types of wireless signals may include Wi-Fi, Bluetooth, ultrawideband (UWB), infrared (IR), ultrasound, and RFID signals.

As a non-limiting example, a patient support apparatus with a location tag assembly may transmit and/or receive UWB and IR signals which may be used by the plurality of wireless locating nodes 230 for precisely locating the patient support apparatus 210.

In one or more alternative implementations, a given patient support apparatus may include one or more RFID readers which enable to detect RFID tags within a detection range of the RFID reader. As a non-limiting example, a given patient support apparatus may include RFID readers that may detect wristbands with RFID transponders or tags when the patient occupies the bed, or when medical staff wears RFID badges. It will be appreciated that other configurations may be possible. The RFID readers may be part of a location tag assembly or connected thereto.

### Mobile Tag

The real-time localization communication system 200 comprises the plurality of mobile wireless tags 220, which may also be referred to as portable identification devices (PID), mobile tags, beacons or transponders. In FIG. 2, the plurality of mobile wireless tags 220 includes a first wireless tag 222 and a second wireless tag 224, however it should be noted that the plurality of mobile wireless tags 220 can include dozens, hundreds, or more wireless tags without departing from the scope of the present technology.

Each of the plurality of mobile wireless tags 220 is a device configured for portable use within an environment and operable to transmit and/or receive one or more types of wireless signals, which enables at least approximately locating the wireless tag. In one or more implementations, a given mobile wireless tag may be attached to equipment (e.g. pumps, patient monitoring devices, ventilator, bedside diagnostic equipment, carts, etc.), objects (e.g., containers, fluid bags) and/or may be carried by a human (e.g., in the form of a wearable badge, card, attached to a clothing piece, or a wearable device such as a wristband). A non-limiting example of a wireless tag in a wristband 1800 is shown in FIG. 18.

Each wireless tag 222, 224 of the plurality of wireless tags 220 is associated with a respective unique identifier (ID). A given wireless tag may include one or more radiofrequency transmitters and/or receivers. It should be understood that the number and types of wireless technologies used in a wireless tag is not limited and different wireless technologies may be combined. For example, the first wireless tag 222 may use Bluetooth and optionally IR transceivers, while the second wireless tag 224 may use RFID transceivers, and a third wireless tag (not shown) may use IR, UWB and Bluetooth transceivers without departing from the scope of the present technology. The term “transceiver” as used herein should be understood to encompass either a transmitter or a receiver, or as a combination transmitter-receiver, unless stated otherwise.

In one or more implementations, the first wireless tag 222 is implemented as an iBeacon tag that periodically transmits its unique identifier via Bluetooth. As a non-limiting example, the iBeacon mobile tag may use Bluetooth low energy (BLE) to broadcast unique identifiers (e.g., MAC ID). It will be appreciated that the first wireless tag 222 may transmit further information such as signal strength related parameters (e.g., RSSI at 1 m) if applicable.

In implementations where the first wireless tag 222 includes an UWB transceiver, the first wireless tag 222 may be configured to determine a distance between the given wireless tag and a wireless locating node, patient support apparatus or other type of device configured with an UWB transceiver and processing capabilities. The distance may be determined using two-way ranging techniques or time difference of arrival (TDoA). The determined distance may then be transmitted via another type of wireless signal (e.g. Bluetooth).

It will be appreciated that alternatively, the distance may be determined by the other device with the UWB transceiver and processing capabilities (e.g. wireless locating node) instead of the given wireless tag with the UWB transceiver.

In one or more other implementations, the first wireless tag 222 may include an infrared receiver for confirming presence of the given wireless tag within an area (e.g., room), and a Bluetooth transceiver configured for transmitting and/or receiving BLE iBeacon signals. In one or more alternative implementations, the first wireless tag 222 may include ultrasonic transceivers for confirming presence of the given wireless tag within an area (e.g. room) using ultrasonic waves, and a Bluetooth transceiver configured for transmitting and receiving BLE iBeacon signals.

It will be appreciated that the second type of wireless signal (e.g., infrared or ultrasonic signal) has limited propagation through physical barriers, which enables determining presence of the at least one mobile tag within a specific area such as a room.

In one or more implementations, the first wireless tag 222 may be implemented using Bluetooth (e.g., iBeacon), UWB, and infrared technologies. In such implementations, infrared may be used to detect presence of the wireless tag within an area (e.g., room), UWB may be used to determine the distance with cm precision, and BLE may be used to transmit the distance and/or other data to the wireless locating node.

In some implementations, a given one of the plurality of wireless tags 220 may also be associated with information such as product or asset information, sensor data, timestamp information, location information (if at a fixed location), status or condition data, authentication or security data, configuration parameters, and usage history or logs. As a non-limiting example, a given wireless tag may comprise an accelerometer to detect different types of movements and/or falls.

In one or more implementations, the second wireless tag 224 may be a passive tag comprising one or more radio-frequency identification (RFID) transponders. As a non-limiting example, the given wireless tag 224 may be attached to clothes worn by the patient or medical worker or may be worn by the patient or medical worker (e.g., on a wristband). The second wireless tag 224 may also be attached to medical equipment (e.g., bed accessories, pumps, IV bag, mattress) which may be itself secured to the bed or may be separate from the bed (e.g., pumps, vital signs monitors, ventilators, defibrillators, suction apparatus, ultrasound imaging systems, etc.). Detection of the second wireless tag 224 enables real-time identification of the patient, visitor, staff or medical equipment associated with the second wireless tag 224 (for example in a table or database), which enables real-time identification and spatial localization of the associated medical device or asset, which in turn facilitates inventory control, asset tracking, and clinical workflow management.

In some implementations, a given one of the plurality of wireless tags 220 may include an accelerometer configured to detect acceleration forces along one or more axes (e.g., x; y; z). The accelerometer generates output signals representative of changes in velocity or orientation of the given wireless tag relative to a reference frame. The output signals may be processed to determine movement characteristics or positional information of the patient or the medical equipment to which it is attached. In such implementations, the accelerometer motion data may be used to determine if the given wireless tag 222, 224 is in movement when the distance and/or the proximity of the given wireless tag 222, 224 to the terminal is determined, which may be used to adjust the determined distance or proximity (e.g., if the patient/equipment with the mobile tag is moving quickly) and/or provide an indication that the mobile tag is in movement to a user viewing the location.

As a non-limiting example, the given one of the plurality of wireless tags 220 including the accelerometer may be attached to the torso of the patient, and the acceleration data may be used to determine a position of the patient (e.g., lying down on their back, sitting, standing, ambulating, reclining, bending, side-lying, seated upright, slouched) as well as transitions between positions including falls. In some implementations, the accelerometer may be implemented as a Microelectromechanical Systems (MEMS) and may be further configured to detect vital signs of the patient.

The given wireless tag 222, 224 may communicate the data to a computing device (e.g., server 240) using the transceivers (e.g. via Bluetooth). It will be appreciated that presence in the bed may be determined using the distance of the wireless tag from the bed/wireless locating node (e.g. via UWB), may be determined using confirmation from the bed load cells, or directly by the bed (in implementations where the given wireless tag includes RFID technology and the bed includes an RFID reader).

In one or more implementations of the present disclosure, a given one of the plurality of wireless tags 220 may be at least approximately located within a room, or its proximity to one or more patient support apparatuses, wireless terminals or other mobile tags may be determined, as will be explained hereinafter.

The real-time localization communication system 200 comprises the plurality of wireless locating nodes 230 (not separately numbered).

### Wireless Locating Node

The wireless locating node 230, which may also be referred to as a wireless terminal, wireless node, wireless anchor, headwall, wireless stationary node, or locator, is a computing device with components configured for transmitting and/or receiving a plurality of types of wireless signals for locating different types of mobile wireless tags attached to beds, equipment, personnel, visitors and/or patients.

The wireless locating node 230 comprises a housing with one or more processing units, one or more memories, and one or more communication interfaces including wireless antennas for transmitting and/or receiving the different types of wireless signals. A given wireless locating node may be positioned within a zone of a room for detecting patient support apparatuses and mobile tags. As a non-limiting example, a wireless locating node 230 may be fixed to a ceiling or a wall of a room, and a room with a plurality of beds may include a plurality of wireless locating nodes. A given wireless terminal may be associated with a unique wireless terminal ID.

In one or more implementations, the wireless locating nodes 230 may include one or more of Wi-Fi, UWB, Bluetooth, ultrasonic, RFID, and IR modules to receive and/or transmit wireless signals.

In one or more implementations, the wireless locating node 230 may include an ESP32 SoC microcontroller manufactured by Taiwan Semiconductor Manufacturing Company (TSMC) Limited from Hsinchu Science Park, Taiwan.

In some implementations, the wireless locating node 230 may be connected to a nurse call system of the medical facility and may exchange wireless and/or wired nurse call signals with the plurality of patient support apparatuses 210.

A given wireless locating node 230 may be operatively connected via a wired or wireless connections for exchanging data via different types of networks, such as local networks, nurse call networks, hospital networks, mobile networks and the Internet.

In one or more implementations, the wireless locating node 230 may be used to at least approximately locate a given mobile wireless tag, and transmit the location data via the communication network 290.

In one or more implementations, the wireless locating node 230 comprises wireless modules and is configured for transmitting and/or receiving one or more of: Wi-Fi signals, infrared (IR) signals, ultrasonic signals, ultrawideband (UWB) signals and Bluetooth signals. Additionally, or alternatively, the wireless terminal may use other types of RF protocols including Zigbee, near-filed communication (NFC), cellular, and the like. The wireless locating node 230 may have components similar to the components of location tag assembly 570 of FIG. 5.

In some embodiments, the wireless locating node 230 may exchange data with the patient support apparatuses 210 and the computing devices connected to the communication network 290.

In one or more implementations, Bluetooth, IR, UWB and/or ultrasonic signals may be used to locate personal identification devices (PID) such as tags or patient support apparatuses (e.g., hospital beds) equipped with IR and UWB chips (not shown in FIG. 2), as described in International Patent Publication WO 2022/208437 A1 by the same Applicant, which is herein incorporated by reference in its entirety.

As a non-limiting example, UWB signals may be used to determine distance with one of time difference of arrival (TDoA) and two-way ranging (TWR).

In one or more implementations, Bluetooth signals may be used to locate mobile tags attached to medical equipment, personnel and/or patients. In one or more implementations, a given wireless locating node may be configured for detecting wireless signals emitted by a given mobile tag and for determining wireless signal parameters including a received signal strength indicator (RSSI) and transmitting the wireless signal parameters to the server 240 executing the RTLS engine 252.

In one or more alternative implementations, Bluetooth signals could be used to determine a distance between a given one of the plurality of wireless locating nodes 230 and a first mobile tag 222 using Bluetooth channel sounding methods. As a non-limiting example, the method may include transmitting, by the terminal, a sounding packet containing a known reference sequence over a designated Bluetooth channel. The given mobile tag 222 may receive the sounding packet and process the reference sequence to estimate a channel impulse response or phase information indicative of signal propagation characteristics. The mobile tag 222 generates a response packet including the channel estimation data and a timestamp corresponding to the time of reception, and transmits the response packet to the terminal. Upon receiving the response packet, the wireless node 230 calculates a time-of-flight or phase shift value based on the round-trip time and the reference signal characteristics, and determines an estimated distance between the terminal and the mobile tag using the speed of electromagnetic wave propagation and the computed timing or phase shift information. It will be appreciated that the distance may in some implementations be determined by the given mobile tag 222, while in other implementations it may be determined by another computing device.

In one or more other implementations, a combination of Bluetooth signals and IR signals may be used to locate mobile tags attached to medical equipment, personnel, visitors and/or patients. In such implementations, a given wireless locating node may be configured for: (i) transmitting infrared signals; (ii) detecting Bluetooth signals emitted by mobile tags including a given mobile tag having received the IR signal; (iii) determining wireless signal parameters including the received signal strength indicator (RSSI); and (iv) transmitting the wireless signal parameters to the server 240 executing the RTLS engine 252. It will be appreciated that this enables detecting tags in line of sight of the wireless locating node using IR.

### Server

The real-time localization communication system 200 comprises a server 240. While illustrated as a single server for simplicity, it should be understood that the functionality of the server 240 described herein may be distributed among a plurality of servers.

The server 240 is configured to inter alia: (i) execute a monitoring functionality 244; (ii) execute a real-time locating system (RTLS) engine 252; (iii) execute a Message Queuing Telemetry Transport (MQTT) broker functionality 258; (iv) execute a training procedure of ML models 260; (v) use the ML models 260 for inference.

It should be noted that the training procedure of ML models 260 and inference of ML models 260 does not need to be performed in each and every embodiment of the present technology.

In one or more implementations, the RTLS engine 252 is configured to inter alia: (i) receive respective first signal parameters of a first type of wireless signal transmitted by at least one wireless tag and received by each respective wireless locating node from the at least one mobile tag; (ii) determine, based the respective first signal parameters, a respective signal strength for each respective wireless locating node from the at least one mobile tag; (iii) estimate, based on the respective signal strengths, respective distances between the at least one wireless tag and each respective wireless locating node; (iv) classify, based on the respective estimated distances, a respective proximity of the at least one wireless tag to each respective wireless locating node.

In some implementations, the RTLS engine 252 is configured to inter alia: (i) receive location and distances of patient support apparatuses to respective wireless locating nodes; (ii) associate locations of the patient support apparatuses to zones within a room; (ii) determine the proximity of BLE tags to respective wireless locating nodes; (iii) receive, from the patient support apparatuses 210, indications of detected RFID tags attached to entities (i.e., users and/or equipment); and (iv) associate location of RFID tags with patient support apparatuses.

The RTLS engine 252 is configured to receive one or more of: identification data, location and movement data, temporal data, status and state data and sensor data related to the plurality of tags 222, 224. This enables: (i) mapping the unique IDs of the tagged entities (e.g. patient, staff, equipment) to the room location and hospital information system; (ii) determining dwell time (presence within a defined area); (iii) determining entry and exit events; (iv) determining movement trajectories (transitions between different detection zones); (v) determining sensor-derived data (if tags include sensors such as accelerometers); and (vi) determining inventory and utilization data.

The RTLS engine 252 enables having a real-time representation of the location of beds, patients, staff and medical equipment, which also enables providing security, inventory management and enables optimizing resources allocated to caregiving and enables improving clinical outcomes.

In one or more implementations, the RTLS engine 252 is configured to use rule-based procedures to associate wireless locating nodes to respective tags based on the estimated distances and the classified proximities. Examples of rule-based procedures will be provided hereinafter.

The MQTT broker functionality 258 is configured to ensure the efficient transmission of data from the wireless nodes to the RTLS engine 252, by using the MQTT protocol. An MQTT broker is implemented to manage communication between the different clients of the system. The MQTT protocol enables using a multiplicity of providers which reduces technological dependency, enables high transparency which facilitates adaptation to future requirements, and provides lightweight and optimized protocol specifically designed for Internet of Things (IoT) applications. The MQTT broker functionality 258 may be implemented as a microservice. In such implementations, the server 240 (or another server) may act as an MQTT broker, while devices such as the wireless nodes may act as a MQTT client.

In the context of the present disclosure, the MQTT broker functionality 258 is used for transmitting and managing location information from the plurality of tags 220 and/or the plurality of wireless locating nodes 230.

In one or alternative implementations, the MQTT broker functionality 258 may be replaced by one or more of: Apache Kafka, RabbitMQ, WebSocket, AWS IoT Core, Google Cloud IoT Core, ActiveMQ and REST APIs without departing from the scope of the present technology.

In one or more implementations, the MQTT broker functionality 258 and the RTLS engine 252 may be provided by a single server. In one or more other implementations, the MQTT broker functionality 258 and the RTLS engine 252 may be distributed among a plurality of servers.

In some implementations, the server 240 is configured to determine actions to execute in response to one or more conditions related to tag or device locations being satisfied. Non-limiting examples of conditions include: detection of tags associated with medical staff within a proximity zone of a bed, detection of tags associated with patients outside of a patient support apparatus, lack of detection of tags associated with medical staff after a threshold time, detection of medical equipment within a zone, detection of care interaction events via detection of a plurality of tags, detection of unauthorized entities by detecting unregistered tags, and the like.

Non-limiting examples of actions executed in response to conditions being satisfied include generating an audible, visual, or haptic alert at the patient support apparatus or at a remote computing device, transmitting a notification to a nurse call system or facility workflow management platform, triggering automated workflows (care protocol, safety alerts, staff notifications, billing events), activating or deactivating one or more bed functions (e.g., siderail locking, bed status functions, bed exit detection, or height adjustment inhibition), activating fall risk protocols, adjusting bed settings to patient preferences, configured room environment, initiating a logging event recording the identity of the detected tag with the satisfied condition, a timestamp, and the apparatus identifier, establishing a communication session between the patient support apparatus and a proximate clinical device, trigger rounding reminders and schedules.

In one or more implementations, the server 240 is configured to provide a graphical user interface (GUI) via the monitoring functionality 244, which enables to receive and display data related to the subsystems and components of the patient support apparatus 210 (e.g., bed status, bed exit, alerts, etc.) and transmit control data to at least some of the subsystems and components of the patient support apparatus 210 via the GUI. In the context of the present disclosure, the monitoring functionality 244 provides monitoring data and location data of the plurality of patient support apparatuses 210, the plurality of mobile tags 220, and the plurality of wireless locating nodes 230. It will be appreciated that the GUI may be accessed by client devices such as client device 282 and workstation computer 284.

The server 240 is configured to process numerical data and transform the numerical data into multiple forms of visual representations. The visual representations may include, but are not limited to, line plots, bar graphs, pie charts, scatter plots, heat maps, histograms, and color-coded diagrams. Additionally, the server 240 is configured to generate visualizations like 2D and 3D graphs, time lapses, interactive plots, and real-time data visualizations. The generated visual data is then displayed as a part of the GUI, which can be accessed by users through computing devices and/or display units such as the nurse station computer 356, and the client device 282, for example via a monitoring application (not shown). The monitoring application 244 enables users to interact with the visual data, as a non-limiting example via functionalities like zooming, panning, and selecting specific data points for a more detailed view, or program functionalities of the patient support apparatus 210.

The workstation computer 284 is a centralized computing system configured to manage and access patient information, location data of the patient support apparatuses 210, the plurality of wireless nodes 230 and the plurality of mobile tags 220, coordinate care, and to enable and facilitate communication among healthcare professionals in a healthcare facility. The workstation computer 284 is configured to access and/or store electronic medical records (EMRs), scheduling and tracking patient appointments, integrating with hospital-wide communication and monitoring systems, assisting in medication management, and providing tools for reporting and analytics.

In one or more implementations of the present technology, the workstation computer 284 is configured to execute a monitoring application (not shown). The monitoring application may be executed as a stand-alone software application, as an application or may be accessible via a browser application (not shown).

In one or more implementations, the workstation computer 284 is configured to: (i) receive data from the patient support apparatuses 210, the wireless locating nodes 230, and the server 240; and (ii) transmit data the patient support apparatuses 210, the wireless locating nodes 230, and the server 240. Optionally, in some implementations, the workstation computer 284 may receive and/or transmit data to the mobile tags 220.

As a non-limiting example, the workstation computer 284 may receive and display, via monitoring application, data related to the subsystems and components of the patient support apparatus 210 (e.g., bed status, bed exit, alerts, etc.), the wireless mobile tags 220, the wireless locating nodes 230, the real-time location data and transmit control data to at least some of the subsystems and components of the patient support apparatus patient support apparatus 210, the wireless mobile tags 220, and the wireless locating nodes 230.

The system further comprises other servers 270 which are configured to host and manage one or more facility-level data services, including: (i) an electronic medical records (EMR) server operable to store, retrieve, and transmit patient clinical data comprising medical histories, diagnostic records, medication profiles, care plans, and clinical notes associated with individual patients admitted to or monitored within the medical facility; and (ii) an admission, discharge, and transfer (ADT) server operable to maintain and transmit patient census data comprising admission records, room and bed assignments, discharge statuses, and inter-unit transfer events, the ADT server being further configured to generate and broadcast ADT event notifications to other networked systems upon a change in patient location or care status within the medical facility, whereby other servers 270 provide the system with access to patient identity, location, and clinical context data in support of the operational functions described herein.

The system 200 comprises client devices 282 (only one instance shown in FIG. 2), associated with one or more users (not shown), such as medical personnel. As such, the client device 282 can sometimes be referred to as a “computing device”, “end user device” or “client computing device”.

The client device 282 may be a tablet used by one or more users, such as medical staff. It will be appreciated that the client device 282 may be implemented as a server, a desktop computer, a laptop, a smartphone, a tablet, and the like without departing from the scope of the present technology.

The one or more communication networks 290 may include one or more of wireless local area networks (WLANs), wired local area networks (LANs), personal area networks (PANs), nurse call systems, wide area networks (WANs), cellular networks, internet of things (IoT) networks, mesh networks, virtual private networks (VPNs), optical fiber networks, and digital health platforms.

How a given communication link 295 (not separately numbered) between the patient support apparatuses 210, the server 240, the workstation computer 284, and the client device 282 is implemented will depend inter alia on how each computing device is implemented. It will be appreciated that each given communication link 295 may be of a different type (e.g., wired or wireless) and may form or be connected to a different type of network of the one or more communication networks 290.

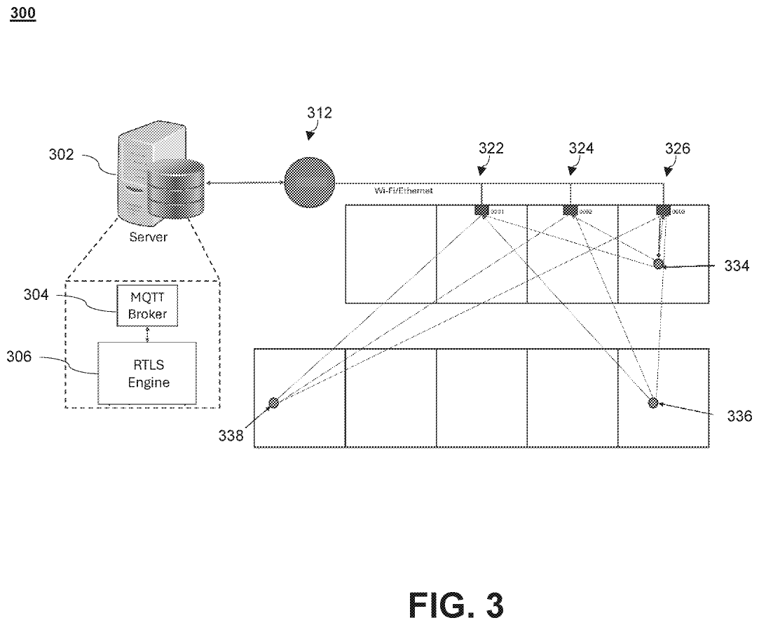

FIG. 3 illustrates a schematic diagram of an exemplary first environment 300 implementing a real-time locating system (RTLS) with mobile wireless tags in a medical facility in accordance with one or more non-limiting implementations of the present disclosure.

In this embodiment, the first environment 300 comprises wireless locating nodes 322, 324, 326 with IR transceivers configured to confirm presence within a specific room, such as room 0003 where the third wireless locating node 326 is located. The first environment 300 further comprises mobile tags 334, 336, 338, where each tag 334, 336, 338 includes a Bluetooth Low Energy (BLE) chip configured to broadcast wireless (e.g., Bluetooth iBeacon) signals, which are detectable by the plurality of wireless locating nodes 322, 324, 326 deployed throughout the facility.

Each room, including room 0003, and selected corridor zones are equipped with a wireless locating node 322, 324, 326 referred to herein as an “MQTT-Client” or “Anchor.” The wireless locating nodes 322, 324, 326 are connected to the local network infrastructure via Wi-Fi or Ethernet, which may include connectivity through access points or Power over Ethernet (PoE) switches 312.

The wireless locating nodes 322, 324, 326 are configured to receive BLE signals emitted by the tags 334, 336, 338. The infrared receiver in each wireless locating node 322, 324, 326 is further configured to detect presence specifically within rooms equipped with IR transmission beams, thereby identifying occupancy at a high level of spatial granularity.

The wireless locating nodes 322, 324, 326 are configured to transmit distance-related or proximity-related data, including Received Signal Strength Indicator (RSSI) values and IR detection status, to a central server 302 using the MQTT protocol. The central server 302 executes a real-time location system (RTLS) engine 306 that aggregates and processes data from all nodes.

With respect to position interpretation, the BLE-based iBeacon signals are analyzed by assessing variations in the received signal strength, which are used to characterize the distance between the tag and each node. The resulting proximity classification includes categories such as “Far,” “Close,” “Out,” or “In Room.” The wireless locating node 322, 324, 326 each transmit an infrared signal within the room; if the tag 334, 336, 338 receives this signal, the node registers the patient's presence within the specific room, such as room 0003. It will be appreciated that there may be two or more possible categories of classification.

Based on the analysis of BLE and IR data, the RTLS engine 306 executed on the server 302 determines the tag location proximity. If the tag has received the IR signal and is concurrently detected as being within the room by BLE-based analysis, the RTLS engine 306 identifies the tag 334 as being located in room 0003. If the IR signal is not received but BLE proximity suggests spatial adjacency, the RTLS engine 306 may identify the tag 336 as being close to but outside room 0003 with a label such as, for example, “Close 0003 (Out).” If the RSSI indicates a more distant location, the tag 338 may be classified as being far from and outside room 0003 with a label such as, for example, “Far 0001 (Out).”

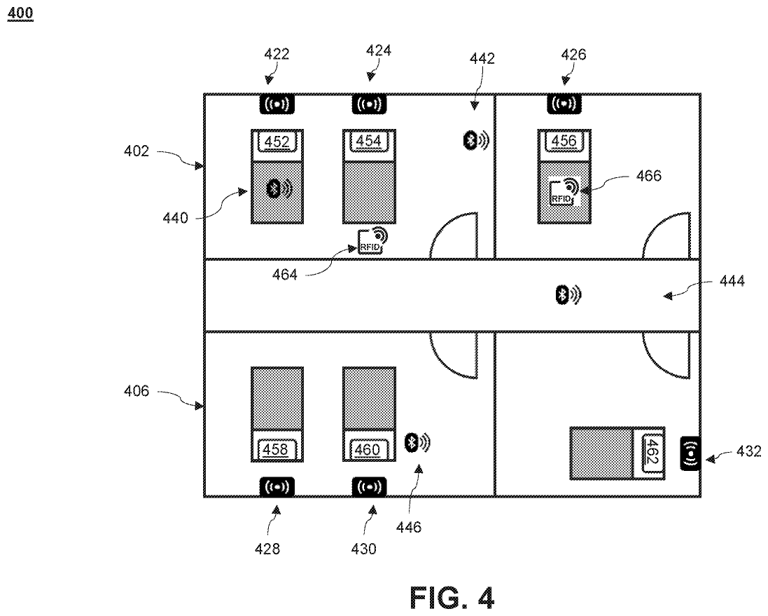

FIG. 4 illustrates a schematic diagram of a second environment 400 in a medical facility where different locations of mobile tags attached to patients or equipment are shown in accordance with one or more non-limiting implementations of the present disclosure. The second environment 400 is a typical environment in which implementations of the present technology may be implemented. In FIG. 4, there is one wireless node 422, 424, 426, 428, 430, 432 per clinical position with a corresponding bed 452, 454, 456, 458, 460 and 462 such that multiple wireless nodes may be present in the same room, such as for example shared room 402 including wireless nodes 422, and shared room 406 including wireless nodes 428, 430, where each wireless node is positioned to cover a zone including a corresponding bed 458, 460 efficiently. Each bed 452, 454, 456, 458, 460 and 462 has a respective location tag assembly with a UHF RFID reader configured to detect RFID tags in proximity of beds with a range of about 1 m. Each bed 452, 454, 456, 458, 460 and 462 is configured to detect a RFID tag and transmit the ID of the RFID tag such that it can be associated with an entity (e.g. medical equipment, patient or staff) by a computing system. There is a first RFID tag 464 attached to medical equipment which has been detected by bed 454, and a second RFID tag 466 detected by bed 456 which is attached to a patient. The beds 454, 456 determine the ID of each tag

For purposes of localization of the BLE mobile tags 440, 442, 444, 446, only one wireless node may be used per room, and nodes should be installed in zones where monitoring is desired, such as hospital wings, storage closets, hallways, etc. To optimize the localization of tags, nodes may be distributed as evenly as possible, with a constant distance between them to ensure optimal coverage.

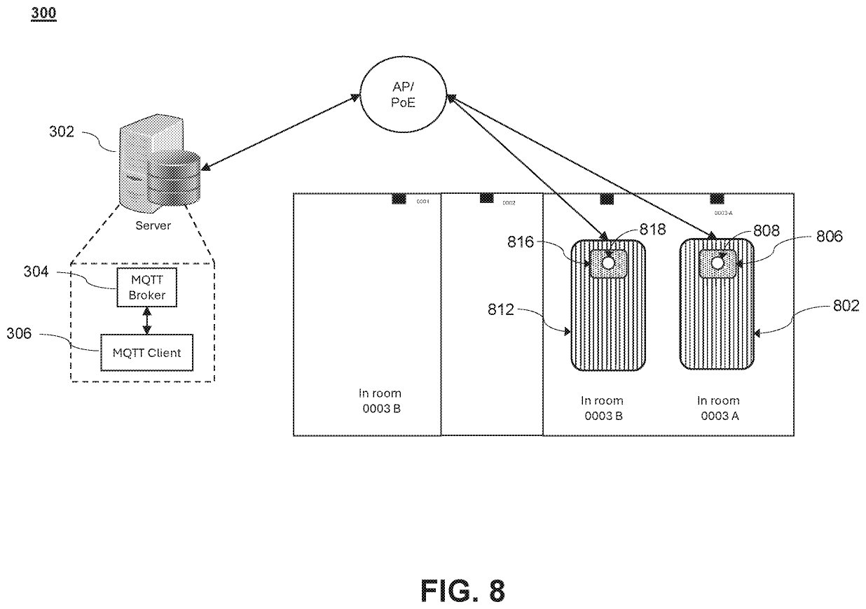

With reference to FIG. 8, there is illustrated a schematic diagram of a location system 800 using RFID in accordance with one or more non-limiting implementations of the present disclosure.

In this embodiment, two patient support apparatuses are located in the same room in two different zones: a first bed 802 is located in room 3A and second bed 812 is located in room 3B. Each bed 802, 812 includes at least one RFID reader 808, 818 coupled to its structure to detect a patient wearing a RFID mobile tag and/or medical equipment with a RFID mobile tag attached thereto.

Each RFID reader 806, 816 comprises an antenna and a control circuit (not separately numbered) configured to generate an interrogation signal and process a response signal. During operation, the RFID reader 806, 816 controls the antenna via the control circuit to emit an electromagnetic field at a predetermined carrier frequency via the antenna, thereby establishing a field of detection, also referred to as radiative coupling region or interrogation zone. A respective RFID tag 808, 818 located within the field of detection includes an RFID tag antenna and RFID integrated circuit configured to interact with the electromagnetic field. In the case of a passive RFID tag, energy from the field generated by the RFID reader 806, 816 is rectified to provide operating power to the integrated circuit of the RFID tag 808, 818. The integrated circuit of the RFID tag 808, 818 modulates an electrical load connected to the tag antenna in accordance with stored identification data, to produce a corresponding variation in the electromagnetic field through backscatter modulation. Each RFID reader 806, 816 is configured to receive, via the antenna, the modulated field, and the RFID reader 806, 816 is configured to demodulate, via the receiver, the variation to recover the encoded data. The RFID reader processes the recovered data using the control circuit to identify the RFID tag. The controller of the bed receives the ID of the RFID tag and may transmit the ID to one or more connected computing devices.

In the implementations discussed hereinafter, the RFID tag 808, 818, which may be attached to a patient or to medical equipment, is equipped with at least one RFID transceiver, and may be detected in real-time by one or more RFID readers 806, 816 located on, or in proximity to, a given patient support apparatus. The given patient support apparatus, which is equipped with communication interfaces (e.g., Bluetooth, IR, UWB, etc.) may then be located in real-time by the wireless locating node. As a result, the patient or medical equipment may be located by association with the wireless locating terminal in real-time.

In some implementations, at least one RFID reader 806, 816 may be integrated within or fixed to a structure of the patient support apparatus to detect RFID tags worn by a subject (e.g., patient or medical staff). At least one RFID reader 806, 816 may be positioned such that the RFID tag associated with the patient is within its field of detection (also referred to as interrogation zone). In one or more implementations, the RFID reader 806, 816 may be an ultra-high frequency (UHF) RFID reader with a range of detection of approximately 1 meter. It will be appreciated that the RFID reader may also detect RFID tags attached to medical equipment or other objects that are within the field of detection.

In one or more implementations, the RFID reader may be located substantially centrally on the patient support apparatus, for example at a foot end of the backrest panel, or at head end or the foot end of a seat panel of the patient support apparatus such that a RFID tag on the wristband of the patient is within its field of detection.

In one or more other implementations, the at least one RFID reader may be integrated in proximity to or into the headboard, the footboard, and/or the side rails of the patient support apparatus.

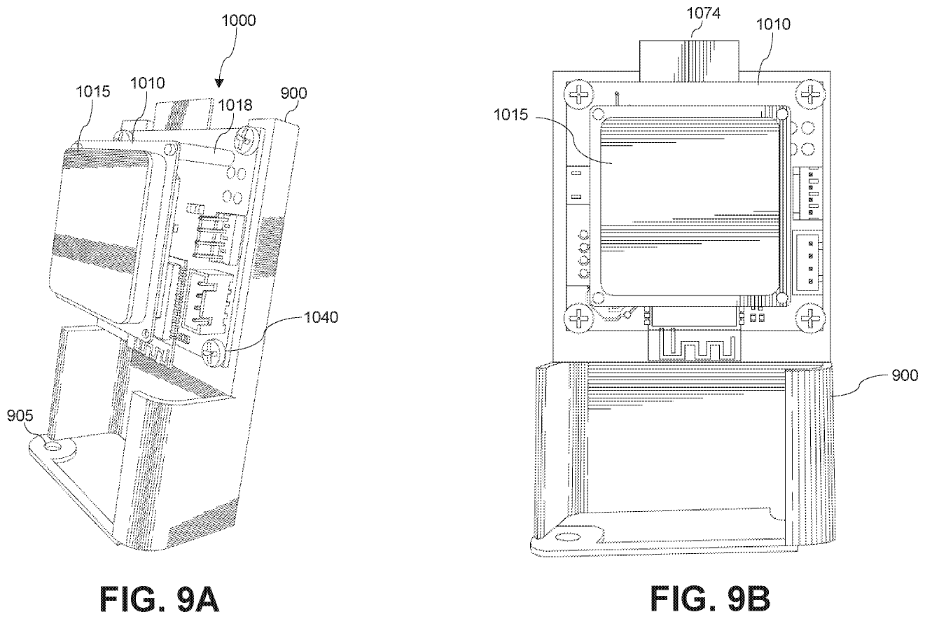

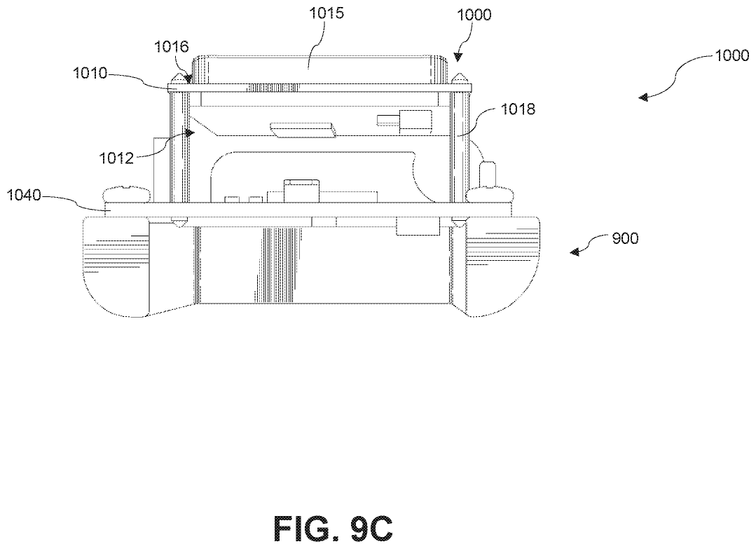

FIG. 9A, FIG. 9B and FIG. 9C show a housing 900 comprising a location tag assembly 1000. The housing 900 is an enclosure insertable into a footboard of hospital bed 100 (FIG. 1). The housing 900 has a generally elongated body sized and shaped to receive the location tag assembly 1000, which can be fixed to the housing 900 for example by using screws. In this embodiment, the housing 900 is made of a material similar to the footboard (not shown) for seamless integration with the footboard in the hospital bed. The housing 900 defines an opening 905 at its bottom to enable fastening the housing 900 to the footboard.

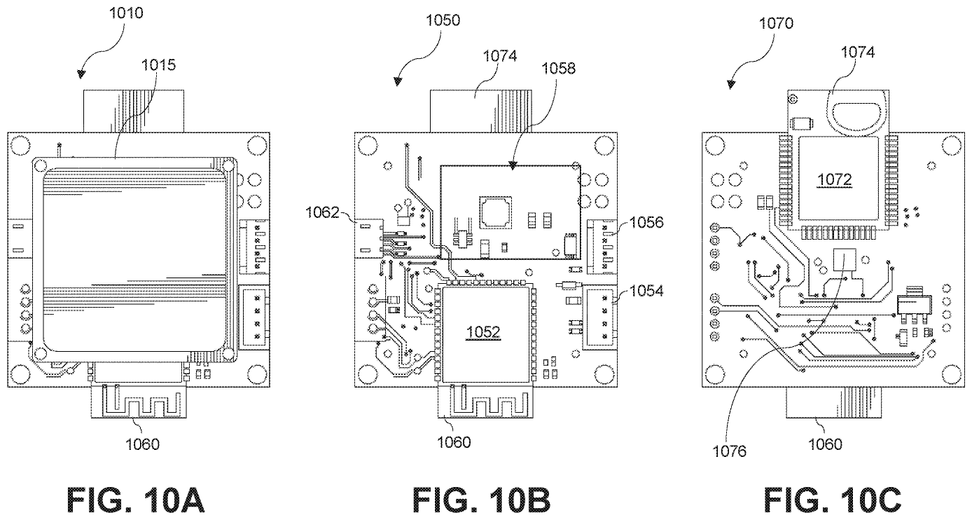

The location tag assembly 1000 will now be described in more detail, with reference to FIG. 9A, FIG. 9B, FIG. 9C, FIG. 10A, FIG. 10B and FIG. 10C.

The location tag assembly 1000 is provided on two stacked boards, a top board 1010 and bottom board 1040. This location tag assembly architecture enables maximizing signal reception and transmission and minimizes interference by strategic positioning of the various components. The two stacked boards are each implemented as printed circuit boards (PCBs).

In this embodiment, the location tag assembly 1000 comprises the top board 1010 disposed above the bottom board 1040. The top board 1010 is mounted on the bottom board 1040 using mounting posts 1018 on the four corners of each of the boards 1010, 1040. It will be appreciated that alternative techniques or fasteners may be used to mount the top board 1040 above the bottom board 1040.

The top board 1010 of the location tag assembly 1000, when mounted in a removable component of the patient support apparatus such as the footboard, is positioned to face the patient support surface of the bed, while the bottom board 1040 is located outwardly and positioned closer to the exterior side of the footboard.

The top board 1010 defines a top board inner surface 1012, and a top board outer surface 1016. The top board outer surface 1016 faces outwardly, while the top board outer surface 1012 faces inwardly, i.e., towards the bottom board 1040. The top board outer surface 1016 includes the RFID antenna 1015.

The RFID antenna 1015 is mounted on the top board 1010 such that the RFID antenna 1015 faces the patient support surface when the location tag assembly 1000 is mounted to the footboard and the footboard is mounted on the hospital bed 100. In one embodiment, the RFID antenna 1015 is an ultra-high-frequency (UHF) antenna.

In some implementations, the RFID antenna 1015 is a circularly polarized RFID antenna. The circularly polarized RFID antenna 1015 is configured to provide an axial ratio of 3 dB or less across its operational beamwidth, such that a linearly polarized RFID tag disposed within the beamwidth is excitable by the radiated wave regardless of the rotational orientation of the tag antenna relative to the reader antenna, which eliminates polarization nulls that would otherwise arise from angular misalignment between the reader and the tag and bounding the polarization mismatch loss to no more than 3 dB across all tag orientations in the plane perpendicular to the propagation axis.

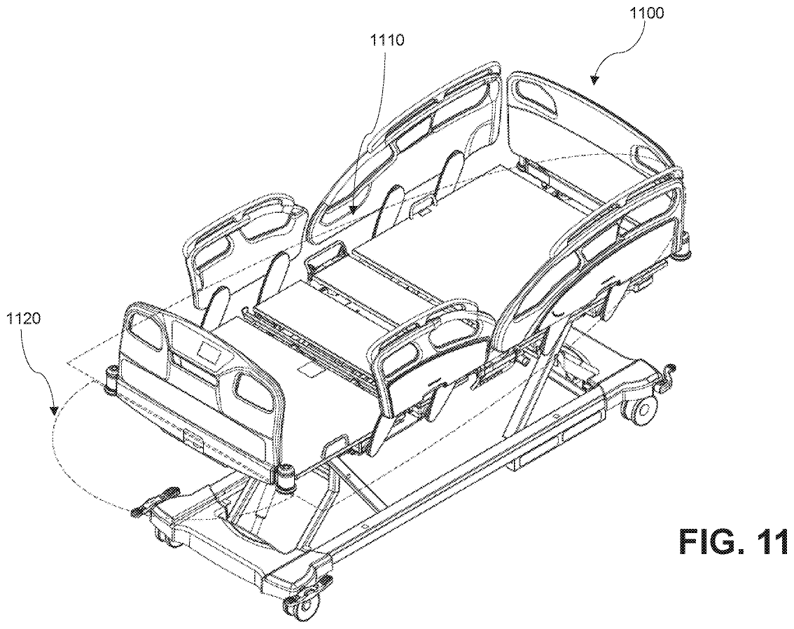

In this configuration, as shown in FIG. 11, the electromagnetic waves radiated by the RFID antenna 1015 on bed 1100 define a first or main detection zone 1110 that covers at least a major portion of the bed such that an RFID tag attached to the patient is detected when the patient is positioned in the bed. In some implementations, the detection zone covers a length of about 2 m towards the patient. The RFID antenna 1015 also defines a secondary or smaller detection zone 1120 facing away from the patient and the interior of the bed 1100 (i.e., directed outwards), which enables to detect RFID tags on medical staff and/or on medical equipment in close proximity of the footboard of the bed.

The bottom board 1040 defines a bottom board inner surface 1050, and a bottom board outer surface 1070. The bottom board inner surface 1050 faces the top board inner surface 1012, while the bottom board outer surface 1070 faces away from the top board inner surface 1012 and towards the exterior of the bed 100.

The bottom board outer surface 1070 includes an UWB module 1072 including a UWB antenna 1074 and an IR receiver 1076. The IR receiver 1076 is positioned to face outwardly to detect IR signals transmitted towards the bed. The IR receiver 1076 may be a wide angle 140-degree infrared receiver. It will be appreciated that the positioning of the wide-angle infrared receiver enables maximizing IR signal detection from IR transmitters, such as IR transmitters included in wireless locating nodes.

The bottom board inner surface 1050 includes a combined Wi-Fi and BLE module 1052 including a combined Wi-Fi/BLE antenna 1060, RFID connectors 1054, a LIN and power connector 1056, and a power management circuit 1058.

The RFID connectors 1054 enable connecting the RFID antenna 1015 of the top board 1010 to the bottom board 1040.

The power management circuit 1056 is configured to transmit and manage power to the components of the location tag assembly 1000.

The LIN and power connector 1056 connects to the LIN connectors and power circuit of the removable barrier (e.g. footboard). In this embodiment, the removable footboard has connectors with electrical connections such that when the removable footboard is inserted into the patient support apparatus, the footboard and the location tag assembly 1000 are powered by the patient support apparatus and operatively connected to the databus of the patient support apparatus. In such implementations, the footboard and the location tag assembly 1000 may transmit data to and receive data from the removable footboard. In alternative implementations, the location tag assembly 1070 may include a battery (not shown) connected to the power management circuit 1058 for powering the location tag assembly.

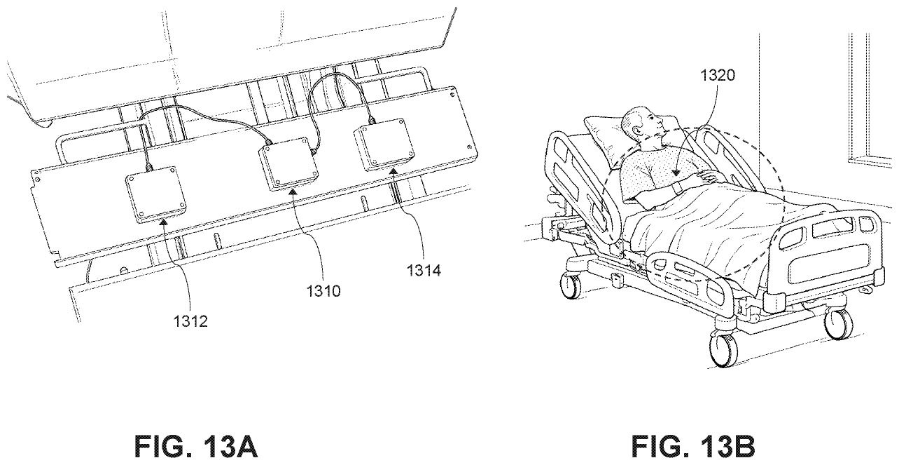

In some implementations, the patient support apparatus may include two RFID readers each positioned such that the RFID tag associated with the patient can be detected accurately, for example if the patient wears the tag on a wristband on a left limb or a right limb. As a non-limiting example, the RFID readers may be located at opposite sides of the patient support apparatus (e.g. left and right sides or head end and foot end) or at different sides of the patient support apparatus, as shown in FIG. 13A.

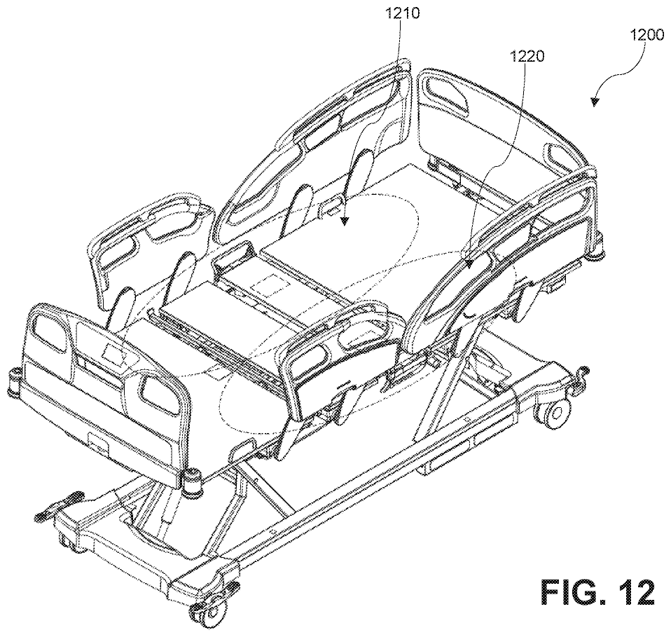

FIG. 13A is a perspective view of a central panel of a hospital bed taken from below showing a left side RFID reader 1312 and right side RFID reader 1314 operatively connected to a controller 1310. Thus, the left side RFID reader 1312 may detect a wristband with RFID tag worn on the left hand of the patient, and the right side RFID reader 1314 may detect a wristband with a RFID tag worn on the right hand of the patient. FIG. 12 shows a perspective view of a bed 1200 with a representation of an approximate left RFID zone 1220 and an approximate right RFID zone 1210 with respective left and right RFID readers (not numbered) under the seat panel. In such embodiments, the left detection zone defined by the left side RFID reader 1312 and the right detection zone defined by the right side RFID reader 1314 may intersect, and the left side RFID reader 1312 and the right side RFID reader 1314 are configured to emit respective signals asynchronously, i.e., one at a time in a successive manner.

In one or more implementations, as shown in FIG. 13B, one or more passive RFID tags may be provided on a wristband 1320 worn by the patient. The wristband 1320 may include for example two RFID transponders located at opposite sides of the wristband such that at least one RFID transponder can be detected in various orientations of the hand of the patient when the patient is positioned within the patient support apparatus e.g., in a lying or sitting position. It will be appreciated that using two RFID transponders on the wristband provides advantages with respect to signal detection and mitigation of electromagnetic interference caused by the wearer's hand. When the wristband 1320 is worn on the wrist, the presence of the hand and its associated biological mass (comprising muscle, bone, and fluid) introduces a dielectric load that may attenuate or reflect electromagnetic signals, particularly in the ultra-high frequency (UHF) range commonly used in passive RFID systems. This attenuation can result in a reduction in effective read range or intermittent detection of the transponder.

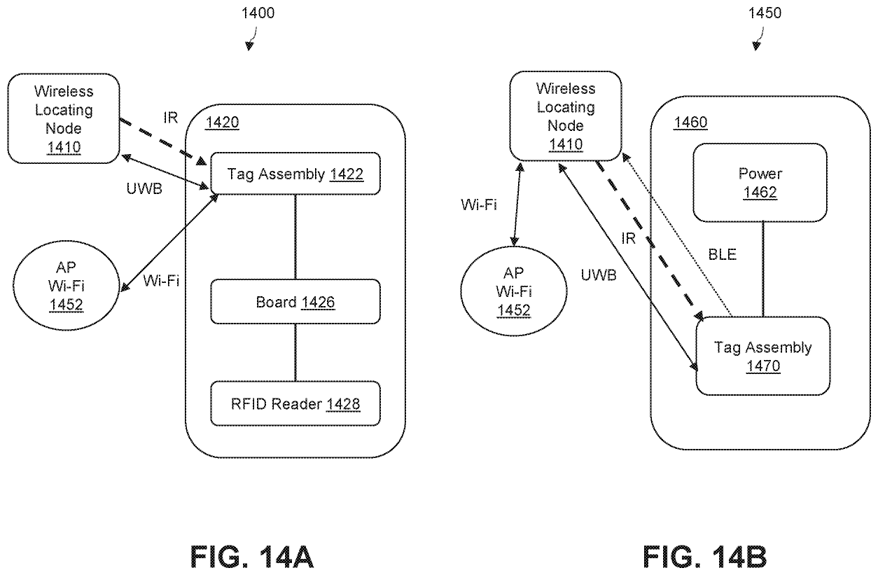

FIG. 14A illustrates a first configuration 1400 of a hospital bed with an integrated location tag assembly 1422 operatively connected to a main board 1426 and a RFID reader 1428 on the bed. The RFID reader 1428, which includes a RFID antenna and processing circuit (not shown), is configured to detect RFID tags worn by a patient and transmit an indication of the detection of the RFID tags to the board 1426 and the tag assembly 1422. The presence of the hospital bed within the room is detected via an IR signal transmitted to the tag assembly 1422 and the distance is calculated using UWB signals transmission between the tag assembly 1422 and the wireless locating node 1410. The tag assembly 1422 may communicate the detected presence of the patient via Wi-Fi to a Wi-Fi access point (AP) 1452. Thus, the patient may be located by locating the bed and the location and distance of the bed in the room may be accurately determined.

FIG. 14B illustrates a schematic diagram of a second configuration 1450 of a hospital bed 1460 with a tag assembly 1470 attached thereto. The hospital bed 1460 may be a third-party hospital bed to which the tag assembly 1470 is retrofitted. In this embodiment, the tag assembly 1470 is implemented as a RFID reader and RTLS tag 1470 using IR, UWB and BLE signals. The RFID reader in the tag assembly 1470 detects the RFID tags worn by a patient via RFID, the presence of the hospital bed 1460 in the room where the wireless locating node 1410 is located is detected via an IR signal transmitted to the tag assembly 1470 and the distance between the tag assembly 1470 and the wireless locating node 1410 is calculated based on exchanged UWB signals. The tag assembly 1470 may communicate the detected presence of the patient and the calculated distance via BLE to the wireless locating node 1410. The wireless locating node 1410 communicates the distance of the bed in the room and an indication of the presence of the patient to the Wi-Fi AP 1452 via Wi-Fi.

In one or more implementations, the presence of the patient in the patient support apparatus may be detected or further confirmed by detecting the weight of the patient on the patient support apparatus via sensors (e.g., load cells, camera, etc.).

In one or more other implementations, an RFID tag similar to the RFID tag worn by a patient or staff may be attached to the mattress positioned on the bed, which enables locating and associating the mattress with the patient support apparatus using the same techniques described herein.

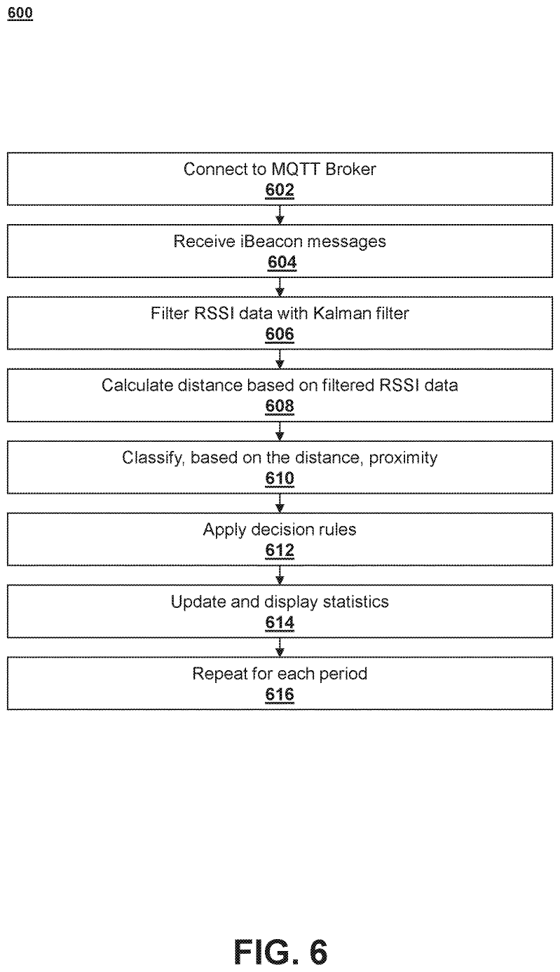

FIG. 6 is a flow chart of a first procedure 600 for locating mobile tags in accordance with one or more non-limiting implementations of the present disclosure. The first procedure 600 is for locating Bluetooth tags (e.g., iBeacon tags).

The first procedure 600 is executed by one or more computing devices including one or more processing units operatively connected to a non-transitory storage medium. In one or more implementations, the computing device executing the first procedure 600 includes the server 240 of FIG. 2.

At processing step 602, the at least one processing unit connects to the MQTT Broker. The at least one processing unit establishes a connection with the MQTT broker to receive iBeacon messages from MQTT clients. In one or more implementations, the MQTT broker is executed by the server 240 of FIG. 2.

At processing step 604, the at least one processing unit receives iBeacon messages. Messages from iBeacon tags are received via the MQTT channel, containing the necessary information (e.g., RSSI values). The iBeacon messages are messages received by the wireless nodes and broadcast by mobile BLE tags. The iBeacon messages include wireless signal parameters detected by the wireless nodes. The wireless signal parameters include RSSI values.

At processing step 606, the at least one processing unit filters RSSI data with a Kalman Filter. The RSSI (Received Signal Strength Indicator) data values are filtered to reduce fluctuations and improve calculation accuracy. In alternative implementations, other types of filters may be used instead of Kalman filters.

At processing step 608, the at least one processing unit calculates a distance based on the filtered RSSI data. The distances between the mobile BLE tags and the nodes (i.e., anchors) are calculated using a signal loss model based on the RSSI data values.

At processing step 610, the at least one processing unit classifies proximity based on the distance. The nodes (anchors) are classified into proximity categories such as one of Very Close, Close, and Far based on the calculated distances.

At processing step 612, the at least one processing unit applies decision rules. The decision rules are applied to determine which wireless node to associate with each iBeacon based on proximity data. Non-limiting examples of decision rules will be described hereinafter with reference to FIGS. 12A to 12C.

At processing step 614, the at least one processing unit updates and optionally causes display of statistics. The processed statistics and information are updated and displayed on the user interface, such as the nurse workstation computer 284 of FIG. 2.

At processing step 616, the at least one processing unit repeats the procedure 600 for each period for each configured iBeacon.

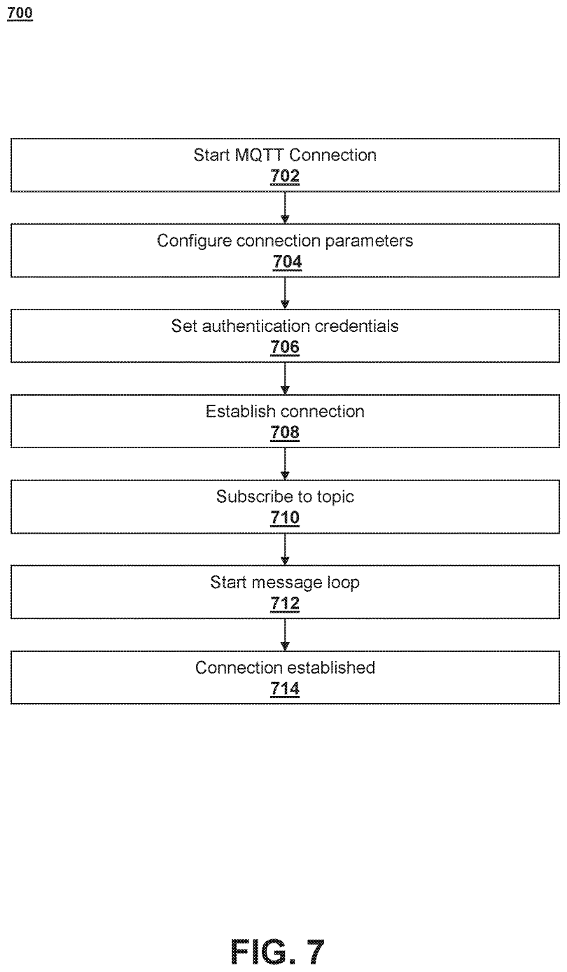

FIG. 7 is a flow chart of a procedure 700 for connecting to a Message Queuing Telemetry Transport (MQTT) broker in accordance with one or more non-limiting implementations of the present disclosure.

In one or more implementations, the MQTT broker may be the MQTT broker 258 executed by server 240 of FIG. 2. The procedure 700 is executed by at least one processing unit of a computing device, such as a processing unit of each of the plurality of wireless locating nodes 230 of FIG. 2.

At processing step 702, the at least one processing unit initiates the MQTT connection by preparing the necessary computing resources to establish a secure connection with the MQTT broker. The at least one processing unit launches the MQTT client and invokes the requisite functions to configure the client's connection settings.

At processing step 704, the at least one processing unit proceeds to configure the connection parameters, including the broker's address, the communication port, and the TLS certificate, to ensure the security of the communication channel through SSL/TLS encryption, which protects the data exchanged between the client, server and the MQTT broker.

At processing step 706, the at least one processing unit sets the authentication credentials, including the username and password, which are required by the MQTT broker. The server provides these credentials to the MQTT client via the username_pw_set function, thereby ensuring that only authorized clients can establish a connection with the broker.

At processing step 708, the at least one processing unit establishes the connection with the MQTT broker using the previously configured settings, including the broker address, port, and authentication information. Upon successful connection, the at least one processing unit is able to interact with the broker.

At processing step 710, the at least one processing unit subscribes to one or more predefined MQTT topics, enabling the at least one processing unit to receive messages published to these topics. The at least one processing unit subscribes to a specific topic using the subscribe method, thereby enabling the processing unit to receive relevant data, such as iBeacon information, published to the subscribed topic.

At processing step 712, the at least one processing unit initiates a message processing loop, wherein a thread is started to continuously listen for incoming messages from the MQTT broker. The at least one processing unit processes the received messages as they arrive in real-time, ensuring that it can interact with the broker and process the transmitted data without delay.

At processing step 714, the MQTT connection is fully established, and the at least one processing unit is capable of receiving and processing real-time messages from the broker, thereby enabling efficient and secure data exchange.

TABLE 1 provides an example of four terminals identified via MAC address which have been classified in terms of proximity according to their estimated distances to a mobile BLE tag:

| Terminal MAC ID | Distance | Proximity |
|---|---|---|
| 17:00:00:00:00:00 | 0.5623 | Very Close |
| 1D:00:00:00:00:00 | 0.8796 | Very Close |
| 2A:00:00:00:00:00 | 3.8796 | Close |
| C1:00:00:00:00:00 | 5.6234 | Far |

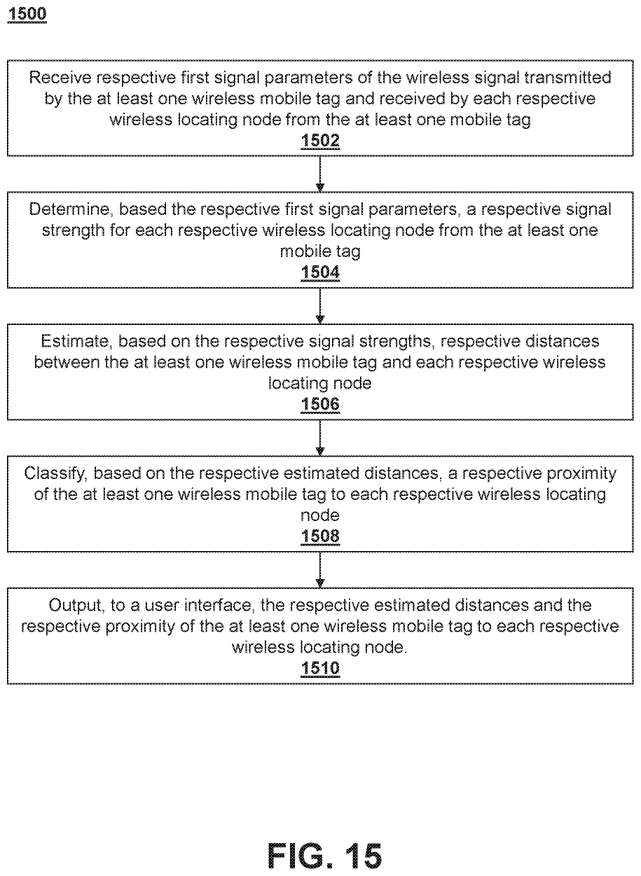

With reference to FIG. 15, there is illustrated a flowchart of a method 1500 of locating wireless mobile tags in accordance with one or more non-limiting implementations of the present technology. The method 1500 may be executed within the environment of FIG. 2.

In one or more implementations, the method 1500 is executed by at least one processing unit. The at least one processing unit is operatively connected to a non-transitory storage medium storing computer-readable instruction thereon. The at least one processing unit, upon executing the computer-readable instructions, is configured to or operable to execute method 1500.

In one or more implementations, the method 1500 is executed by the RTLS engine 252 executed by the server 240. The server 240 is operatively connected to the plurality of wireless nodes 230 over the communication network 290. The plurality of wireless nodes 230 are configured for receiving wireless signals transmitted by the plurality of wireless tags 220.

The method 1500 begins at processing step 1502.

At processing step 1502, the at least one processing unit receives respective first signal parameters of the wireless signals transmitted by the at least one wireless mobile tag and received by each respective wireless locating node from the at least one mobile tag.

Each of the plurality of wireless tags 220 transmits wireless signals, which are detected by the wireless locating nodes 230 in range. Each wireless locating node 230 in range measures first wireless signal parameters of the wireless signals received by nodes in range.

In one or more implementations, each of the plurality of wireless tags 220 includes a respective Bluetooth beacon and is associated with an identifier.

In one or more implementations, the first wireless signal parameters may include one or more of power-based parameters (RSSI, RSRP, path-loss, including received power metric, carrier frequency and channel number, antenna gain/pattern descriptors, receiver gain, environment model terms). As a non-limiting example, the wireless signal may be a Bluetooth Low Energy signal, and the wireless signal parameters include at least the RSSI and a unique identifier of the tag.

At processing step 1504, the at least one processing unit determines, based the respective first signal parameters, a respective signal strength for each respective wireless locating node from the at least one mobile tag.

In implementations using RSSI, the RTLS engine 252 determines the signal strength at each wireless node of the plurality of wireless nodes 230 with respect to each wireless tag of the plurality of wireless tags 220 in range. The RTLS engine 252 applies a filter on the respective RSSI to obtain the respective signal strength. In one or more implementations, the RTLS engine 252 applies a Kalman filter on the respective RSSI to obtain the respective signal strength.

At processing step 1506, the at least one processing unit estimates, based on the respective signal strengths, respective distances between the at least one wireless mobile tag and each respective wireless locating node.

It will be appreciated that the signal strength is used to determine the distance in implementations using power-based parameters, and in implementations using signal propagation time measurement parameters or phase difference measurement across frequencies parameters, the distance may be determined based on the signal propagation time measurement parameters and the phase difference measurement parameters without using signal strength.

In some implementations, where the wireless nodes and mobile tags are equipped with infrared or ultrasonic receivers and transmitters, the at least one processing unit may receive second type of signal data (e.g., infrared signal or ultrasonic signal data), which may indicate if a given tag was detected by a wireless node within a delimited zone, as such signals do not propagate through physical barriers. In combination with the first wireless signal strength parameters, this enables directly discriminating signal strength data from tags not located in the same room as the wireless node, thus optimizing computing resources when calculating distances and proximity.

In one or more implementations, the RTLS engine 252 estimates the distance between a given mobile tag and each respective wireless node 230 based on the respective signal strength using a path loss exponent in a propagation model. The RTLS engine 252 may use the RSSI at 1 meter in addition to the respective signal strength in the form of a filtered RSSI in a path loss propagation model to calculate the distance estimate.

At processing step 1508, the at least one processing unit classifies, based on the respective estimated distances, a respective proximity of the at least one wireless mobile tag to each respective wireless locating node.

In one or more implementations, the RTLS engine 252 classifies the proximity of a given mobile tag to each wireless node based on the respective distance being above, equal to and/or below one or more thresholds.

As a non-limiting example, estimated distances below a first threshold (e.g. 2 m) may be classified as very close, estimated distances between a first threshold and a second threshold (e.g., between 2 m and 5 m) may be classified as close, and estimates distances above a third threshold (e.g., 5 m) may be classified as far. The RTLS engine 252 calculates the proximity for each mobile wireless tag.

In some implementations, the at least one processing unit may transmit, with regard to each wireless locating node, the respective estimated distances and the respective proximity of the at least one wireless mobile tag to each respective wireless locating node.

In one or more other implementations, the method 1500 comprises processing step 1510.

At processing step 1510, the at least one processing unit outputs, to a user interface, the respective estimated distances and the respective proximity of the at least one wireless mobile tag to each respective wireless locating node.

In one or more implementations, the respective estimated distances and the respective proximity of the at least one wireless mobile tag to each respective wireless locating node is transmitted to a client device, such as the workstation computer 284 and/or client device 282 for display.

In one or more other implementations, the respective estimated distances and/or the respective proximity of at least one wireless mobile tag to each respective wireless locating node are transmitted for output into alternative forms (e.g., image, video, audio, etc.)

The method 1500 then ends.

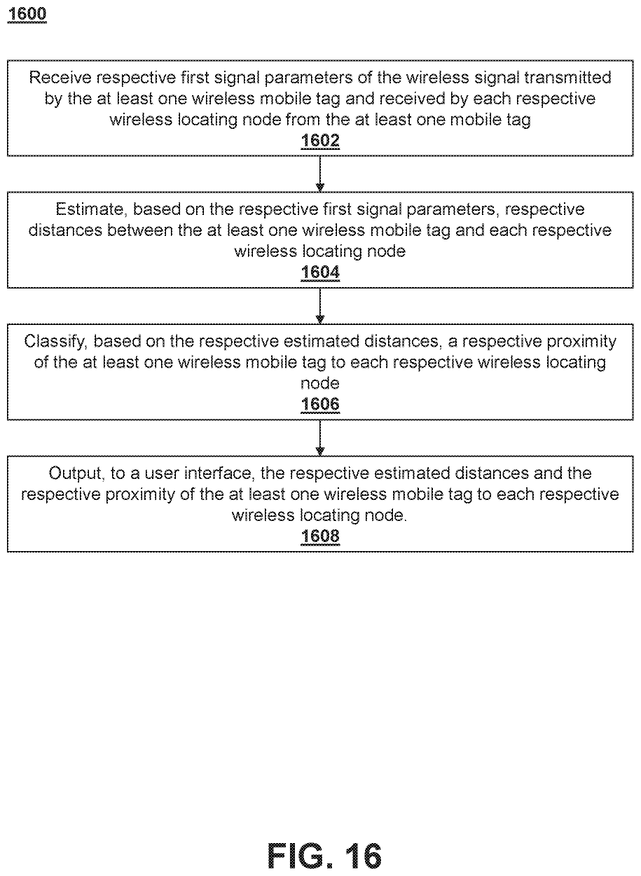

With reference to FIG. 16, there is illustrated a flowchart of a method 1600 of locating wireless mobile tags in accordance with one or more non-limiting implementations of the present technology. The method 1600 may be executed within the environment of FIG. 2.

The method 1600 is similar to method 1500 of FIG. 15, however the distances are determined based on one of signal propagation time measurement parameters or phase difference measurement across frequencies parameters, without using signal power parameters (e.g., RSSI).

In one or more implementations, the method 1600 is executed by at least one processing unit. The at least one processing unit is operatively connected to a non-transitory storage medium storing computer-readable instruction thereon. The at least one processing unit, upon executing the computer-readable instructions, is configured to or operable to execute method 1600.

In one or more implementations, the method 1600 is executed by the RTLS engine 252 executed by the server 240. The server 240 is operatively connected to the plurality of wireless nodes 230 over the communication network 290. The plurality of wireless nodes 230 are configured for receiving wireless signals transmitted by the plurality of wireless tags 220.

The method 1600 begins at processing step 1602.

At processing step 1602, the at least one processing unit receives respective first signal parameters of the wireless signals transmitted by the at least one wireless mobile tag and received by each respective wireless locating node from the at least one mobile tag.

Each of the plurality of wireless tags 220 transmits wireless signals, which are detected by the wireless locating nodes 230 in range. Each wireless locating node 230 in range measures first wireless signal parameters of the wireless signals received by nodes in range.

In one or more alternative implementations, the first wireless signal parameters may include signal propagation time measurement parameters (time of flight (ToF), and round-trip time (RTP) related parameters including time of departure/arrival, timestamp resolution, clock offset and drift, signal bandwidth and sampling, etc.)

In one or more implementations, the first wireless signal parameters may include phase difference measurement across frequencies parameters that enable determining room-level distances (e.g., Frequency Domain Phase Difference of Arrival (FD-PDOA), phase-based ranging, Space Domain Phase Difference of Arrival (SD-PDOA), etc.). Phase difference measurement across frequencies parameters may include one or more of: carrier frequency, frequency separation between tones, number of measurements frequencies, phase resolution, signal bandwidth, and update rate.

At processing step 1604, the at least one processing unit estimates, based on the respective first signal parameters, respective distances between the at least one wireless mobile tag and each respective wireless locating node.

In one or more implementations, the at least one processing unit estimates, based on one of signal propagation time measurement parameters and phase difference measurement across frequencies parameters, the respective distance between the at least one wireless mobile tag and each respective wireless locating node.

In some implementations, where the wireless nodes and mobile tags are equipped with infrared or ultrasonic receivers and transmitters, the at least one processing unit may receive second type of signal data (e.g., infrared signal or ultrasonic signal data), which may indicate if a given tag was detected by a wireless node within a delimited zone, as such signals do not propagate through physical barriers. In combination with the first wireless signal parameters, this enables directly discriminating signal parameters from tags not located in the same room as the wireless node, thus optimizing computing resources when calculating distances and proximity.

At processing step 1606, the at least one processing unit classifies, based on the respective estimated distances, a respective proximity of the at least one wireless mobile tag to each respective wireless locating node.

In one or more implementations, the RTLS engine 252 classifies the proximity of a given mobile tag to each wireless node based on the respective distance being above, equal to and/or below one or more thresholds.

As a non-limiting example, estimated distances below a first threshold (e.g. 2 m) may be classified as very close, estimated distances between a first threshold and a second threshold (e.g., between 2 m and 5 m) may be classified as close, and estimates distances above a third threshold (e.g., 5 m) may be classified as far. The RTLS engine 252 calculates the proximity for each mobile wireless tag.

At processing step 1608, the at least one processing unit outputs, to a user interface, the respective estimated distances and the respective proximity of the at least one wireless mobile tag to each respective wireless locating node.

In one or more implementations, the respective estimated distances and the respective proximity of the at least one wireless mobile tag to each respective wireless locating node is transmitted to a client device, such as the workstation computer 284 and/or client device 282 for display.

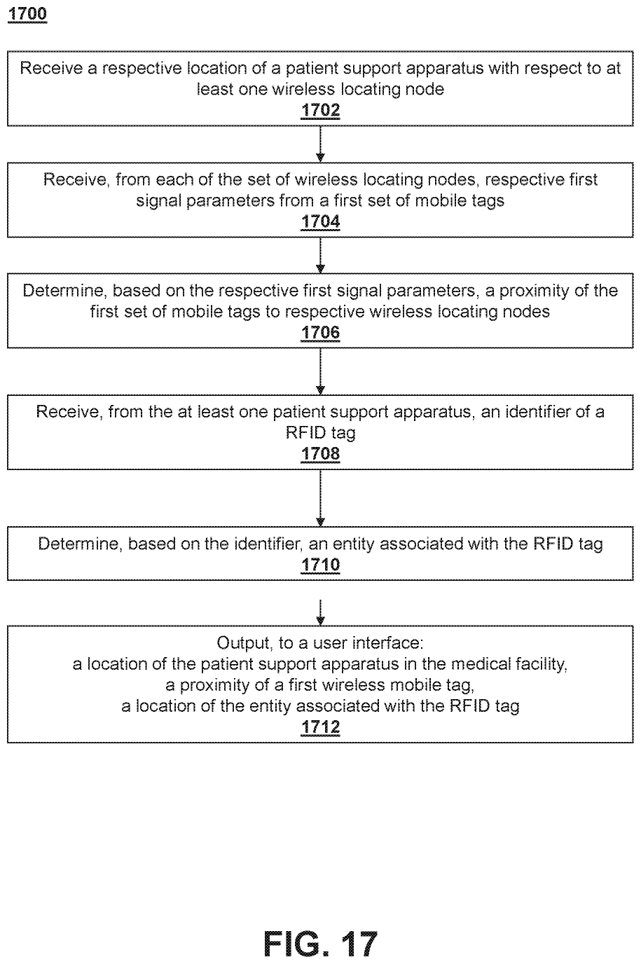

With reference to FIG. 17, there is illustrated a flowchart of a method 1700 of locating wireless mobile tags in accordance with one or more non-limiting implementations of the present technology. The method 1600 may be executed within the environment of FIG. 2.

The mobile tags include a first set of mobile tags and a second set of mobile tags. The first set of mobile tags includes BLE mobile tags, and the second set of mobile tags includes RFID tags.

In some implementations, the method 1700 may be executed by a computing device such as the server 240, the server 240 being operatively connected to a patient support apparatus 210 and a wireless locating node 2230.

The method 1700 may be executed in real-time or near real-time.

According to processing step 1702, the at least one processing unit receives a respective location of a patient support apparatus with respect to at least one wireless locating node.

In one or more implementations, the at least one patient support apparatus determines its own location by confirming presence within a delimited area using infrared and by calculating a distance with clinical precision (cm-level) by exchanging UWB signals with the wireless locating node.

According to processing step 1704, the at least one processing unit receives, from each of the set of wireless locating nodes, respective first signal parameters from a first set of mobile tags.

The first set of mobile tags includes at least one mobile tag, such as an iBeacon tag. The set of wireless locating nodes acquires the signal parameters of signals broadcast by the iBeacon tags.

According to processing step 1706, the at least one processing unit determines, based on the respective first signal parameters, a proximity of the first set of mobile tags to the respective wireless locating nodes.

The at least one processing unit executes method 150 to determine the proximity of the first set of mobile tags with respect to the wireless locating nodes.

According to processing step 1708, the at least one processing unit receives, from the at least one patient support apparatus, an identifier of a RFID tag.

The RFID antenna of the patient support apparatus detects a RFID tag by transmitting an interrogation signal and detecting a RFID tag by receiving a response signal therefrom, the response signal including a unique ID. The RFID tag may be associated with an entity such as a patient, medical staff or with an object or medical equipment. The patient support apparatus transmits, to the at least one processing unit, the unique ID of the detected RFID tag. The at least one processing unit receives the unique ID of the tag and the unique ID of patient support apparatus.

According to processing step 1710, the at least one processing unit determines, based on the identifier, an entity associated with the RFID tag.

The at least one processing unit correlates the unique ID of the RFID tag with the associated entity (e.g., patient, staff, medical equipment, etc.). In some implementations, the at least one processing unit queries a database to determine the entity associated with the unique ID of the RFID tag.

The at least one processing unit associates the entity with the patient support apparatus, which enables locating the entity with near clinical grade precision since the presence of the patient support apparatus within a specific room and the distance of the patient support apparatus from the wireless locating node are known.

In some implementations, the at least one processing unit receives, from the patient support apparatus, the detection information of the RFID tag with time stamps at predetermined intervals, which enables tracking the time spent at given locations.

The at least one processing unit may, for example, transmit the information to the workstation computer 285 and/or the other servers 270 (e.g., ADT server) which enables retrieving information about the entity associated with the RFID tag. The at least one processing unit receives information about the entity (e.g., patient, staff, medical equipment) and associates the entity with the location of the bed.

According to processing step 1712, the at least one processing unit outputs, to a user interface and a location of the patient support apparatus in the medical facility, a proximity of a first wireless mobile tag, and a location of the entity associated with the RFID tag.

In one or more implementations, the at least one processing unit transmits to a user interface, such as a display of the workstation computer 285, or another computing device, a location of the patient support apparatus in the medical facility, a proximity of a first wireless mobile tag, and a location of the entity associated with the RFID tag. The information may be displayed in a GUI or in table format, such that medical staff may track each of the patient support apparatus, the first wireless tag and the RFID tag in real-time. As a non-limiting example, an identification of the entity (e.g., patient), its location (e.g., room 3B), a detection time stamp (e.g., 2025-08-26 09:56:18.0005617) and a time in bed (e.g., 7.644) may be displayed on the workstation computer 285.

It will be appreciated that RFID tags may be used to locate and track patients in patient support apparatuses (i.e., entry, presence time, exit, time spent), locate and track visitors, locate and track medical staff, locate and track objects and medical equipment (e.g., mattress, pumps, etc.)

In some implementations, thresholds or conditions may be associated with locations, time spent at locations, distances, etc. and the entities. In some implementations, the at least one processing unit is configured to determine and transmit actions to execute in response to one or more conditions related to tag or device locations being satisfied. Non-limiting examples of conditions include: detection of tags associated with medical staff within a proximity zone of a bed, detection of tags associated with patients outside of a patient support apparatus, lack of detection of tags associated with medical staff after a threshold time, detection of medical equipment within a zone, detection of care interaction events via detection of a plurality of tags, detection of unauthorized entities by detecting unregistered tags. The actions may cause the computing device receiving the actions to execute the actions (e.g., alarm, display, record, etc.).

Any of the implementations described herein may be combined with one another in various arrangements or subcombinations, unless otherwise specified.

1. A real-time locating system (RTLS) for use in a medical facility, the RTLS comprising:
a location tag assembly adapted to be mounted to a patient support apparatus, the location tag assembly comprising a first set of wireless transceivers comprising a first wireless transceiver, a second wireless transceiver, at least one radiofrequency identification (RFID) transceiver, and a third wireless transceiver, the location tag assembly being configured for:
determining, using the first wireless receiver and the second wireless transceiver, a distance and a location of the location tag assembly in an area;
transmitting, using the third wireless transceiver, the location and the distance to a computing device;
detecting, using the at least one RFID transceiver, a RFID tag by receiving a response RFID signal therefrom;
determining, based on the response, an identifier (ID) of the RFID tag; and
transmitting, using the third wireless transceiver, the identifier of the RFID tag;
a wireless locating node positioned in the area, the wireless locating node comprising a second set of wireless transceivers comprising a corresponding first wireless transmitter and a corresponding second wireless transceiver, the wireless locating node being configured for:
exchanging signals with the location tag assembly using the corresponding first wireless transmitter and the corresponding second wireless transceiver to enable the location tag assembly to determine the distance and the location;
a computing device connected to the location tag assembly, the computing device being configured for:
receiving, from the location tag assembly using the third wireless transceiver, the distance and the location of the location tag assembly relative to the wireless locating node;
associating the location tag assembly with a location of the wireless locating node in the medical facility to thereby obtain a real-time clinical location of the location tag assembly;
receiving, from the location tag assembly using the third wireless transceiver, an identifier of the RFID tag;
determining, based on the identifier of the RFID tag, an entity associated with the RFID tag;
associating the entity associated with the RFID tag to the location of the location tag assembly; and
outputting, to a user interface, an indication of a location of the location tag assembly and the entity associated with the RFID tag.

2. The RTLS system of claim 1, wherein:
the wireless locating node further comprises a Bluetooth transceiver, the wireless locating node being configured for determining signal parameters of a Bluetooth signal broadcast by a Bluetooth tag, and wherein
the computing device is further configured for:
determining, using the signal parameters of the Bluetooth signal, a proximity of the Bluetooth tag to the wireless locating node; and
determining another entity associated with the Bluetooth tag; and
outputting, to a user interface, an indication of the proximity of the other entity to the wireless locating node.

3. The RTLS system of claim 1, wherein the at least one RFID transceiver is adapted to be mounted below a deck panel of the patient support apparatus.

4. The RTLS system of claim 1, wherein the location tag assembly is removably secured to a barrier of the patient support apparatus.

5. The RTLS system of claim 4, wherein the at least one RFID transceiver is positioned to face a patient support surface of the patient support apparatus when the location tag assembly is secured to the barrier of the patient support apparatus.

6. The RTLS system of claim 5, wherein:
the first wireless transceiver comprises an infrared receiver and the corresponding first wireless transmitter comprises an infrared transmitter;
the second wireless transceiver and the corresponding second wireless transceiver each comprise a respective ultrawideband transceiver; and
the third wireless transceiver comprises a Wi-Fi transceiver.

7. A patient support apparatus comprising:
a frame;
a patient support surface mounted to the frame;
a controller mounted to at least one of the frame and the patient support surface;
a location tag assembly operatively connected to the controller, the location tag assembly comprising:
a first wireless receiver configured for receiving a first type of wireless signal;
a second wireless transceiver configured for transmitting and receiving a second type of wireless signal;
at least one radiofrequency identification (RFID) transceiver configured for transmitting and receiving RFID signals for detecting RFID tags;
a third wireless transceiver configured for transmitting a third type of wireless signal;
the controller being configured for:
determining, using the first wireless receiver and the second wireless transceiver, a distance and presence of the patient support apparatus relative to a wireless locating node;
transmitting, using the third wireless transceiver, the location and the distance to a computing device;
detecting, using the at least one RFID transceiver, a RFID tag by receiving a response RFID signal therefrom;
determining, based on the response, an identifier (ID) of the RFID tag; and
transmitting, using the third wireless transceiver, the identifier of the RFID tag to a computing device.

8. The patient support apparatus of claim 7, wherein the RFID transceiver comprises at least one of: a circular polarization RFID antenna and ultrahigh frequency (UHF) RFID antenna.

9. The patient support apparatus of claim 7, wherein the patient support apparatus comprises a removable footboard selectively couplable to a foot end of the frame, the location tag assembly being located within the removable footboard.

10. The patient support apparatus of claim 9, wherein the first wireless receiver comprises an infrared (IR) receiver, the second wireless transceiver comprises an ultrawideband (UWB) transceiver, and the third wireless transceiver comprises at least one of: a Wi-Fi transceiver and a Bluetooth transceiver.

11. The patient support apparatus of claim 10, wherein the location tag assembly comprises a top board disposed above a bottom board and operatively connected thereto, the top board facing a patient support surface of the patient support apparatus when the removable footboard is coupled to the patient support apparatus, the top board comprising an RFID antenna.

12. The patient support apparatus of claim 11, wherein the bottom board comprises the UWB transceiver, the IR receiver and the Wi-Fi transceiver, the IR receiver facing away from the patient support surface when the removable footboard is coupled to the patient support apparatus.

13. The patient support apparatus of claim 12, wherein the removable footboard comprises at least one connector for operatively connecting the location tag assembly to a databus and a power circuit of the patient support apparatus.

14. A location tag assembly adapted to be mounted to a patient support apparatus, the location tag assembly comprising:
a first wireless receiver configured for receiving a first type of wireless;
a second wireless transceiver configured for transmitting and receiving a second type of wireless signal;
at least one radiofrequency identification (RFID) transceiver configured for transmitting and receiving RFID signals for detecting RFID tags;
a third wireless transceiver configured for transmitting a third type of wireless signal;
at least one processing unit operatively connected to the first wireless receiver, the second wireless transceiver, the third wireless transceiver and the at least one RFID transceiver, the at least one processing unit being configured for:
determining, using the first wireless receiver and the second wireless transceiver, a distance and presence of the patient support apparatus relative to a wireless locating node;
transmitting, using the third wireless transceiver, the location and the distance to a computing device;
detecting, using the at least one RFID transceiver, a RFID tag by receiving a response RFID signal therefrom;
determining, based on the response, an identifier (ID) of the RFID tag; and
transmitting, using the third wireless transceiver, the identifier of the RFID tag to a computing device.

15. The location tag assembly of claim 14, wherein the first wireless receiver comprises an infrared (IR) receiver, the second wireless transceiver comprises an ultrawideband (UWB) transceiver, and the third wireless transceiver comprises at least one of: a Wi-Fi transceiver and a Bluetooth transceiver.

16. The location tag assembly of claim 15, wherein the location tag assembly comprises a top board disposed above a bottom board and operatively connected thereto, the top board facing a patient support surface when the location tag assembly is coupled to the patient support apparatus, the top board comprising an RFID antenna.

17. The location tag assembly of claim 16, wherein the bottom board comprises the UWB transceiver, the IR receiver and the Wi-Fi transceiver, the IR receiver facing away from the patient support surface when the location tag assembly is coupled to the patient support apparatus.

18. The location tag assembly of claim 17, wherein the location tag assembly comprises at least one connector for operatively connecting the location tag assembly to a databus and a power circuit of the patient support apparatus.

19. The location tag assembly of claim 18, wherein the RFID transceiver comprises at least one of: a circular polarization RFID antenna and ultrahigh frequency (UHF) RFID antenna.

20. The location tag assembly of claim 19, wherein the IR receiver comprises a 140-degree wide angle receiver.
